(19) United States
(12) Patent Application Publication
(10) Pub. No.: US 2019/0381098 A1
(43) Pub. Date: Dec. 19, 2019

(54) METHODS OF TREATING MULTIPLE MYELOMA AND PLASMA CELL LEUKEMIA BY T CELL THERAPY

(71) Applicant: Memorial Sloan Kettering Cancer Center, New York, NY (US)

(72) Inventor: Guenther Koehne, New York, NY (US)

(73) Assignee: Memorial Sloan Kettering Cancer Center, New York, NY (US)

(21) Appl. No.: 15/758,566

(22) PCT Filed: Sep. 9, 2016
(86) PCT No.: PCT/US2016/050857

§ 371 Date: Mar. 8, 2018

Related U.S. Application Data
(60) Provisional application No. 62/216,525, filed on Sep. 10, 2015.
(60) Provisional application No. 62/220,641, filed on Sep. 18, 2015.

(51) Int. Cl. A61K 35/17; A61P 35/02; C07K 14/47

(52) U.S. Cl. CPC A61K 35/17; C07K 14/4748; A61P 35/02

(57) ABSTRACT

Disclosed herein are methods of treating multiple myeloma in a human patient in need thereof, comprising administering to the human patient a population of allogeneic cells comprising WT1-specific allogeneic T cells. Also disclosed herein are methods of treating plasma cell leukemia in a human patient in need thereof, comprising administering to the human patient a population of allogeneic cells comprising WT1-specific allogeneic T cells.

### CROSS-REFERENCE TO RELATED APPLICATIONS

This application claims the benefit of U.S. Provisional Patent Application Nos. 62/216,525, filed Sep. 10, 2015, and 62/220,641, filed Sep. 18, 2015, which are incorporated by reference herein in their entireties.

### 1. FIELD

Disclosed herein are methods of treating multiple myeloma in a human patient in need thereof, comprising administering to the human patient a population of allogeneic cells comprising WT1-specific allogeneic T cells. Also disclosed herein are methods of treating plasma cell leukemia in a human patient in need thereof, comprising administering to the human patient a population of allogeneic cells comprising WT1-specific allogeneic T cells.

### 2. BACKGROUND

Plasma cell leukemia (PCL) is a rare and aggressive variant of multiple myeloma with very poor prognosis (Jaffe et al., 2001, Ann Oncol 13:490-491). Secondary and primary plasma cell leukemia (pPCL) are the most aggressive forms of the plasma cell dyscrasias. Primary plasma cell leukemia is defined by the presence of >2×10⁹/L peripheral blood plasma cells or plasmacytosis accounting for >20% of the differential white cell count, and does not arise from pre-existing multiple myeloma (MM) (Jaffe et al., 2001, Ann Oncol 13:490-491; Hayman and Fonseca, 2001, Curr Treat Options Oncol 2:205-216). Secondary PCL (sPCL), however, is a leukemic transformation of end stage MM. pPCL is rare, with only 1-4% of MM patients presenting as pPLC (Gertz, 2007, Leuk Lymphoma 48:5-6; Noel and Kyle, 1987, Am J Med 83:1062-1068; Pagano et al., 2011, Ann Oncol 22:1628-1635; Tiedemann et al., 2008, Leukemia 22:1044-1052). The prognosis of pPCL is very poor, with a median overall survival (OS) of only 7 months with up to 28 percent dying within the first month after diagnosis with standard chemotherapy. Median overall survival is even shorter (1.3 month) when occurs in the context of refractory or relapsing multiple myeloma (sPCL) (Tiedemann et al., 2008, Leukemia 22:1044-1052). There are no curative regimens for primary or secondary PCL. Because of the lack of large prospective series, PCL treatment is based on empiric recommendations or extrapolation of data from multiple myeloma literature. The median survival of primary PCL patients receiving autologous stem cell transplant has been reported at 28 months. Autologous stem cell transplant (auto SCT) is considered primary treatment for PCL. A retrospective CIBMTR (Center for International Blood and Marrow Transplant Research) analysis compared the outcome of the 97 patients who received auto SCT with 50 patients who received allogeneic stem cell transplants (allo SCT) between 1995 and 2006 (Attal et al., 1996, N Engl J Med 335:91-97; Perez-Simon et al., 1998, Blood 91:3366-3371; Saccaro et al., 2005, Am J Hematol 78:288-294). Although the cumulative incidence of relapse at 3 years was significantly lower in the allogeneic group (allo SCT vs auto SCT, 38% vs 61%), TRM (transplant-related mortality) at 3 years was considerably higher in patients who received an allogeneic transplant (allo SCT vs auto SCT, 41% vs 5%). This resulted in a 3 year OS of 64% and 39% for the auto SCT and allo SCT group, respectively (Attal et al., 1996, N Engl J Med 335:91-97; Perez-Simon et al., 1998, Blood 91:3366-3371; Saccaro et al., 2005, Am J Hematol 78:288-294). The treatment of pPCL and sPCL therefore requires innovative treatment approaches incorporating novel modalities to improve outcome.

Treatment of relapsed/refractory multiple myeloma (RRMM) presents a special therapeutic challenge, because of the heterogeneity of disease at relapse and the absence of clear biological-based recommendations regarding the choice of salvage therapies at various time points of disease progression. According to the International Myeloma Working Group criteria, progressive disease (PD) is defined by at least a 25% increase from nadir in the serum paraprotein (absolute increase must be ≥0.5 g/dL) or urine paraprotein (absolute increase must be ≥200 mg/24 hours), or in the difference between involved and uninvolved serum-free light-chain (FLC) levels (with an abnormal FLC ratio and FLC difference >100 mg/L). In patients who lack measurable paraprotein levels (oligo- or nonsecretory myeloma), an increase in bone marrow plasma cells (≥10% increase) or new bone/soft tissue lesions increasing the size of existing lesions or unexplained serum calcium >11.5 mg/dL is used to define disease progression. Relapsed and refractory multiple myeloma is defined as progression of disease while on therapy in patients who achieve minor response (MR) or better, or who progress within 60 days of their last therapy. Patients who never achieve at least a MR to initial induction therapy and progress while on therapy are defined as “primary refractory.” Relapsed multiple myeloma is defined as disease in a myeloma patient who has previously been treated and has evidence of PD as defined above, and who at the time of relapse does not meet the criteria for relapsed and refractory or primary refractory multiple myeloma In addition, high-risk cytogenetics such as del(17p) and t(4;14) are correlated with shortened survival.

Patients with refractory or relapsed and refractory multiple myeloma who have exhausted novel agents have limited options and short expected survival. Although a recent phase 3 MM-003 trial demonstrated significant progression-free and overall survival benefits for pomalidomide plus low-dose dexamethasone vs high-dose dexamethasone in patients who failed bortezomib and lenalidomide. But at updated median follow-up 15.4 months, progression-free survival was only 4.0 vs 1.9 months (HR, 0.50; P<0.001), and median overall survival was only 13.1 vs 8.1 months (HR, 0.72; P=0.009) for this patient population. In the high-risk group, pomalidomide plus low-dose dexamethasone vs high-dose dexamethasone improved progression-free survival in patients with del(17p) (4.6 vs 1.1 months; HR, 0.34; P<0.001), t(4;14) (2.8 vs 1.9 months; HR, 0.49; P=0.028), and standard risk (4.2 vs 2.3 months; HR, 0.55; P<0.001). Overall survival for pomalidomide plus low-dose dexamethasone vs high-dose dexamethasone treatment was 12.6 vs 7.7 months (HR, 0.45; P=0.008) in patients with del(17p), 7.5 vs 4.9 months (HR, 1.12; P=0.761) in t(4;14), and 14.0 vs 9.0 months (HR, 0.85; P=0.380) in standard risk. Overall response rate was higher for pomalidomide plus low-dose dexamethasone vs high-dose dexamethasone in standard risk (35.2% vs 9.7%) and del(17p) (31.8% vs 4.3%) and similar in t(4;14) (15.9% vs 13.3%) (Dimopoulos et al., 2015, Haematologica pii: haematol. 2014.117077, published online Aug. 6, 2015).

Patients with relapsed multiple myeloma have been treated with donor lymphocyte infusions after allogeneic T cell depleted hematopoietic stem cell transplantation (Tyler et al., 2013, Blood 121:308-317).

A protocol is available on the clinicaltrials.gov website (NCT01758328) for a Phase I study of relapsed/refractory multiple myeloma patients and plasma cell leukemia patients who, after allogeneic stem cell transplantation, are to be administered WT1-specific donor (of the stem cell transplant)-derived T cells.

Wilms tumor 1 gene (WT1) was originally identified in the childhood renal neoplasm, Wilms tumor (Call et al., 1990, Cell 60:509-520). The non-mutated form of WT1 was originally categorized as a tumor-suppressor gene with roles in the transcriptional regulation of early growth-factor gene promoters. More recently, WT1 has been described as an oncogene. WT1 is overexpressed in a number of hematologic malignancies including up to 70% of acute myelogenous leukemia (AML), acute lymphoblastic leukemia (ALL), chronic myelogenous leukemia (CIVIL), and myelodysplastic syndrome (Miwa et al., 1992, Leukemia 6:405-409). A high level of WT1 by leukemic blasts in AML is associated with poor response to chemotherapy, a greater risk of disease relapse, and reduced probability of extended disease-free survival. For these reasons, WT1 expression serves as a prognostic marker. Several research groups are using quantitative PCR methods to monitor disease response and minimal residual disease (Miwa et al., 1992, Leukemia 6:405-409; Inoue et al., 1994, Blood 84:3071-3079).

MM cells were also recently shown to overexpress WT1. The expression of WT1 in bone marrow correlates with numerous prognostic factors, including disease stage and M protein ratio (Hatta et al., 2005, J Exp Clin Cancer Res 24:595-599). MM cells are highly susceptible to perforin-mediated cytotoxicity by WT1-specific cytotoxic T lymphocytes (CTL), and WT1 expression is sufficient to induce WT1-specific IFN-γ production by CTL (Azuma et al., 2004, Clin Cancer Res 10:7402-7412). Clinical responses have also been reported with WT1 peptide-based immunotherapy. Following immunization with a synthetic WT1 peptide, marked reductions in the myeloma disease-load in bone marrow and level of M protein in the urine were observed, along with bone scintigram improvement. This partial response to vaccination correlated with expansion of functional WT1-specific CTL (cytotoxic T lymphocyte) and migration of WT1-specific T cells to the bone marrow (Azuma et al., 2004, Clin Cancer Res 10:7402-7412).

Citation of a reference herein shall not be construed as an admission that such is prior art to the present disclosure.

### 3. SUMMARY OF THE INVENTION

The present invention relates to methods of treating WT1 (Wilms Tumor 1)-positive multiple myeloma in a human patient. The present invention further relates to methods of treating WT1-positive plasma cell leukemia in a human patient.

Provided herein are methods of treating WT1-positive multiple myeloma in a human patient in need thereof, comprising administering to the human patient a population of allogeneic cells comprising WT1-specific allogeneic T cells.

In one aspect, the methods of treating WT1-positive multiple myeloma in a human patient in need thereof comprise administering to the human patient a population of allogeneic cells comprising WT1-specific allogeneic T cells, wherein the population of allogeneic cells lacks substantial cytotoxicity in vitro toward antigen presenting cells that are not WT1 peptide-loaded or genetically engineered to express one or more WT1 peptides.

In a specific embodiment, the population of allogeneic cells lyses less than or equal to 15% of unmodified phytohemagglutinin-stimulated lymphoblasts derived from the human patient in an in vitro cytotoxicity assay, thereby lacking substantial cytotoxicity in vitro toward antigen presenting cells that are not WT peptide-loaded or genetically engineered to express one or more WT1 peptides.

In another specific embodiment, the population of allogeneic cells lyses less than or equal to 15% of unmodified phytohemagglutinin-stimulated lymphoblasts derived from the donor of the population of allogeneic cells in an in vitro cytotoxicity assay, thereby lacking substantial cytotoxicity in vitro toward antigen presenting cells that are not WT peptide-loaded or genetically engineered to express one or more WT1 peptides.

In another specific embodiment, the population of allogeneic cells lyses less than or equal to 15% of unmodified HLA-mismatched cells of an EBV BLCL in an in vitro cytotoxicity assay, thereby lacking substantial cytotoxicity in vitro toward antigen presenting cells that are not WT peptide-loaded or genetically engineered to express one or more WT1 peptides.

In another specific embodiment, the population of allogeneic cells lyses less than or equal to 15% of unmodified phytohemagglutinin-stimulated lymphoblasts derived from the human patient in an in vitro cytotoxicity assay, and the population of allogeneic cells lyses less than or equal to 15% of unmodified HLA-mismatched cells of the EBV BLCL in an in vitro cytotoxicity assay, thereby lacking substantial cytotoxicity in vitro toward antigen presenting cells that are not WT peptide-loaded or genetically engineered to express one or more WT1 peptides.

In another specific embodiment, the population of allogeneic cells lyses less than or equal to 15% of unmodified phytohemagglutinin-stimulated lymphoblasts derived from the donor of the population of allogeneic cells in an in vitro cytotoxicity assay, and the population of allogeneic cells lyses less than or equal to 15% of unmodified HLA-mismatched cells of the EBV BLCL in an in vitro cytotoxicity assay, thereby lacking substantial cytotoxicity in vitro toward antigen presenting cells that are not WT peptide-loaded or genetically engineered to express one or more WT1 peptides.

In certain embodiments, the population of allogeneic cells further exhibits lysis of greater than or equal to 20% of antigen presenting cells that are WT1 peptide-loaded in an in vitro cytotoxicity assay. In a specific embodiment, the population of allogeneic cells further exhibits lysis of greater than or equal to 20% of WT1 peptide pool-loaded phytohemagglutinin-stimulated lymphoblasts derived from the human patient in an in vitro cytotoxicity assay. In another specific embodiment, the population of allogeneic cells further exhibits lysis of greater than or equal to 20% of WT1 peptide pool-loaded antigen presenting cells derived from the donor of the population of allogeneic cells in an in vitro cytotoxicity assay. In another specific embodiment, the population of allogeneic cells further exhibits lysis of greater than or equal to 20% of WT1 peptide pool-loaded phytohemagglutinin-stimulated lymphoblasts derived from the human patient in an in vitro cytotoxicity assay, and exhibits lysis of greater than or equal to 20% of WT1 peptide pool-loaded antigen presenting cells derived from the donor of the population of allogeneic cells in an in vitro cytotoxicity assay.

In specific embodiment, the first dose of the population of allogeneic cells is administered within 12 weeks after the diagnosis of the multiple myeloma. In a specific embodiment, the first dose of the population of allogeneic cells is administered between 5 to 12 weeks after the diagnosis of the multiple myeloma.

In various embodiments, prior to the administering of the population of allogeneic cells, the human patient has been administered a therapy for multiple myeloma that is different from said population of allogeneic cells. The therapy can be an autologous hematopoietic stem cell transplantation (HSCT), an allogeneic HSCT, a cancer chemotherapy, an induction therapy, a radiation therapy, or a combination thereof, to treat the multiple myeloma. In a specific embodiment, the autologous HSCT is a peripheral blood stem cell transplant. In a specific embodiment, the allogeneic HSCT is a peripheral blood stem cell transplant. The population of allogeneic cells can be derived from the donor of the allogeneic HSCT or a third-party donor that is different from the donor of the allogeneic HSCT.

In certain embodiments, the therapy is an HSCT.

In specific embodiment, the therapy is an autologous HSCT. In a specific embodiment, the autologous HSCT is a peripheral blood stem cell transplant. In some embodiments, the first dose of the population of allogeneic cells is administered on the day of, or up to 12 weeks after, the autologous HSCT. In a specific embodiment, the first dose of the population of allogeneic cells is administered between 5 to 12 weeks after the autologous HSCT.

In other specific embodiment, the therapy is an allogeneic HSCT. In a specific embodiment, the allogeneic HSCT is a peripheral blood stem cell transplant. In a specific embodiment, the population of allogeneic cells is derived from the donor of the allogeneic HSCT. In another specific embodiment, the population of allogeneic cells is derived from or a third-party donor that is different from the donor of the allogeneic HSCT. In some embodiments, the first dose of the population of allogeneic cells is administered on the day of, or up to 12 weeks after, the allogeneic HSCT. In a specific embodiment, the first dose of the population of allogeneic cells is administered between 5 to 12 weeks after the allogeneic HSCT.

In various embodiments, the human patient has failed the therapy prior to said administering of the population of allogeneic cells. In specific embodiments, the multiple myeloma is refractory to the therapy or relapses after the therapy. In a specific embodiment, the multiple myeloma is primary refractory multiple myeloma. In another specific embodiment, the multiple myeloma is relapsed multiple myeloma. In another specific embodiment, the multiple myeloma is relapsed and refractory multiple myeloma. In specific embodiments, the human patient has discontinued the therapy due to intolerance of the therapy.

In other various embodiments, prior to the administering of the population of allogeneic cells, the human patient has not been administered a therapy for multiple myeloma. In specific embodiment, the first dose of the population of allogeneic cells is administered within 12 weeks after the diagnosis of the multiple myeloma. In a specific embodiment, the first dose of the population of allogeneic cells is administered between 5 to 12 weeks after the diagnosis of the multiple myeloma.

In specific embodiments of the methods of treating WT1-positive multiple myeloma as described above, the administering of the population of allogeneic cells does not result in any graft-versus-host disease (GvHD) in the human patient.

Also provided herein are methods of treating WT1-positive plasma cell leukemia in a human patient in need thereof, comprising administering to the human patient a population of allogeneic cells comprising WT1-specific allogeneic T cells.

In one aspect, the methods of treating WT1-positive plasma cell leukemia in a human patient in need thereof comprise administering to the human patient a population of allogeneic cells comprising WT1-specific allogeneic T cells, wherein the population of allogeneic cells lacks substantial cytotoxicity in vitro toward antigen presenting cells that are not WT1 peptide-loaded or genetically engineered to express one or more WT1 peptides.

In some embodiments, the plasma cell leukemia is primary plasma cell leukemia. In other embodiments, the plasma cell leukemia is secondary plasma cell leukemia.

In a specific embodiment, the population of allogeneic cells lyses less than or equal to 15% of unmodified phytohemagglutinin-stimulated lymphoblasts derived from the human patient in an in vitro cytotoxicity assay, thereby lacking substantial cytotoxicity in vitro toward antigen presenting cells that are not WT peptide-loaded or genetically engineered to express one or more WT1 peptides.

In another specific embodiment, the population of allogeneic cells lyses less than or equal to 15% of unmodified phytohemagglutinin-stimulated lymphoblasts derived from the donor of the population of allogeneic cells in an in vitro cytotoxicity assay, thereby lacking substantial cytotoxicity in vitro toward antigen presenting cells that are not WT peptide-loaded or genetically engineered to express one or more WT1 peptides.

In another specific embodiment, the population of allogeneic cells lyses less than or equal to 15% of unmodified HLA-mismatched cells of an EBV BLCL in an in vitro cytotoxicity assay, thereby lacking substantial cytotoxicity in vitro toward antigen presenting cells that are not WT peptide-loaded or genetically engineered to express one or more WT1 peptides.

In another specific embodiment, the population of allogeneic cells lyses less than or equal to 15% of unmodified phytohemagglutinin-stimulated lymphoblasts derived from the human patient in an in vitro cytotoxicity assay, and the population of allogeneic cells lyses less than or equal to 15% of unmodified HLA-mismatched cells of the EBV BLCL in an in vitro cytotoxicity assay, thereby lacking substantial cytotoxicity in vitro toward antigen presenting cells that are not WT peptide-loaded or genetically engineered to express one or more WT1 peptides.

In another specific embodiment, the population of allogeneic cells lyses less than or equal to 15% of unmodified phytohemagglutinin-stimulated lymphoblasts derived from the donor of the population of allogeneic cells in an in vitro cytotoxicity assay, and the population of allogeneic cells lyses less than or equal to 15% of unmodified HLA-mismatched cells of the EBV BLCL in an in vitro cytotoxicity assay, thereby lacking substantial cytotoxicity in vitro toward antigen presenting cells that are not WT peptide-loaded or genetically engineered to express one or more WT1 peptides.

In certain embodiments, the population of allogeneic cells further exhibits lysis of greater than or equal to 20% of antigen presenting cells that are WT1 peptide-loaded in an in vitro cytotoxicity assay. In a specific embodiment, the population of allogeneic cells further exhibits lysis of greater than or equal to 20% of WT1 peptide pool-loaded phytohemagglutinin-stimulated lymphoblasts derived from the human patient in an in vitro cytotoxicity assay. In another specific embodiment, the population of allogeneic cells further exhibits lysis of greater than or equal to 20% of WT1 peptide pool-loaded antigen presenting cells derived from the donor of the population of allogeneic cells in an in vitro cytotoxicity assay. In another specific embodiment, the population of allogeneic cells further exhibits lysis of greater than or equal to 20% of WT1 peptide pool-loaded phytohemagglutinin-stimulated lymphoblasts derived from the human patient in an in vitro cytotoxicity assay, and exhibits lysis of greater than or equal to 20% of WT1 peptide pool-loaded antigen presenting cells derived from the donor of the population of allogeneic cells in an in vitro cytotoxicity assay.

In specific embodiment, the first dose of the population of allogeneic cells is administered within 12 weeks after the diagnosis of the plasma cell leukemia. In a specific embodiment, the first dose of the population of allogeneic cells is administered between 5 to 12 weeks after the diagnosis of the plasma cell leukemia.

In various embodiments, prior to the administering of the population of allogeneic cells, the human patient has been administered a therapy for plasma cell leukemia that is different from said population of allogeneic cells. The therapy can be an autologous hematopoietic stem cell transplantation (HSCT), an allogeneic HSCT, a cancer chemotherapy, an induction therapy, a radiation therapy, or a combination thereof, to treat the plasma cell leukemia. In a specific embodiment, the autologous HSCT is a peripheral blood stem cell transplant. In a specific embodiment, the allogeneic HSCT is a peripheral blood stem cell transplant. The population of allogeneic cells can be derived from the donor of the allogeneic HSCT or a third-party donor that is different from the donor of the allogeneic HSCT.

In certain embodiments, the therapy is an HSCT.

In specific embodiment, the therapy is an autologous HSCT. In a specific embodiment, the autologous HSCT is a peripheral blood stem cell transplant. In some embodiments, the first dose of the population of allogeneic cells is administered on the day of, or up to 12 weeks after, the autologous HSCT. In a specific embodiment, the first dose of the population of allogeneic cells is administered between 5 to 12 weeks after the autologous HSCT.

In other specific embodiment, the therapy is an allogeneic HSCT. In a specific embodiment, the allogeneic HSCT is a peripheral blood stem cell transplant. In a specific embodiment, the population of allogeneic cells is derived from the donor of the allogeneic HSCT. In another specific embodiment, the population of allogeneic cells is derived from or a third-party donor that is different from the donor of the allogeneic HSCT. In some embodiments, the first dose of the population of allogeneic cells is administered on the day of, or up to 12 weeks after, the allogeneic HSCT. In a specific embodiment, the first dose of the population of allogeneic cells is administered between 5 to 12 weeks after the allogeneic HSCT.

In various embodiments, the human patient has failed the therapy prior to said administering of the population of allogeneic cells. In specific embodiments, the plasma cell leukemia is refractory to the therapy or relapses after the therapy. In specific embodiments, the human patient has discontinued the therapy due to intolerance of the therapy.

In other various embodiments, prior to the administering of the population of allogeneic cells, the human patient has not been administered a therapy for plasma cell leukemia. In specific embodiment, the first dose of the population of allogeneic cells is administered within 12 weeks after the diagnosis of the plasma cell leukemia. In a specific embodiment, the first dose of the population of allogeneic cells is administered between 5 to 12 weeks after the diagnosis of the plasma cell leukemia.

In specific embodiments of the methods of treating WT1-positive plasma cell leukemia as described above, the administering of the population of allogeneic cells does not result in any graft-versus-host disease (GvHD) in the human patient.

In a specific embodiment, the population of allogeneic cells that is administered to the human patient is restricted by an HLA allele shared with the human patient.

In specific embodiments, the population of allogeneic cells comprising WT1-specific allogeneic T cells shares at least 2 out of 8 HLA alleles (for example, two HLA-A alleles, two HLA-B alleles, two HLA-C alleles, and two HLA-DR alleles) with the human patient.

In specific embodiments, the methods of treating WT1-positive multiple myeloma or plasma cell leukemia described herein further comprise prior to the administering step a step of ascertaining at least one HLA allele of the human patient by high-resolution typing.

In various embodiments, the methods of treating WT1-positive multiple myeloma or plasma cell leukemia further comprise prior to the administering step a step of generating the population of allogeneic cells in vitro.

In certain embodiments, the step of generating the population of allogeneic cells in vitro comprises sensitizing (i.e., stimulating) allogeneic cells to one or more WT1, wherein the allogeneic cells comprise allogeneic T cells.

In a specific embodiment, the step of generating the population of allogeneic cells in vitro comprises a step of enriching for T cells prior to said sensitizing.

In specific embodiments, the step of generating the population of allogeneic cells in vitro further comprises, after sensitizing, cryopreserving the allogeneic cells.

In specific embodiments, the methods of treating WT1-positive multiple myeloma or plasma cell leukemia described herein further comprise, before the administering step, steps of thawing cryopreserved WT1-peptide sensitized allogeneic cells, and expanding the allogeneic cells in vitro, to produce the population of allogeneic cells.

In specific embodiments, the methods of treating WT1-positive multiple myeloma or plasma cell leukemia described herein further comprise, before the administering step, a step of thawing a cryopreserved form of the population of allogeneic cells.

In certain embodiments, the step of generating the population of allogeneic cells in vitro comprises sensitizing the allogeneic cells using dendritic cells, cytokine-activated monocytes, peripheral blood mononuclear cells, or EBV-BLCL (EBV-transformed B lymphocyte cell line) cells. In specific embodiments, the step of sensitizing the allogeneic cells using dendritic cells, cytokine-activated monocytes, peripheral blood mononuclear cells, or EBV-BLCL cells comprises loading the dendritic cells, the cytokine-activated monocytes, the peripheral blood mononuclear cells, or the EBV-BLCL cells with at least one immunogenic peptide derived from WT1. In specific embodiments, the step of sensitizing the allogeneic cells using dendritic cells, cytokine-activated monocytes, peripheral blood mononuclear cells, or EBV-BLCL cells comprises loading the dendritic cells, the cytokine-activated monocytes, the peripheral blood mononuclear cells, or the EBV-BLCL cells with a pool of overlapping peptides derived from one or more WT1 peptides.

In certain embodiments, the step of generating the population of allogeneic cells in vitro comprises sensitizing the allogeneic cells using artificial antigen-presenting cells (AAPCs). In specific embodiments, the step of sensitizing the allogeneic T cells using AAPCs comprises loading the AAPCs with at least one immunogenic peptide derived from WT1. In specific embodiments, the step of sensitizing the allogeneic T cells using AAPCs comprises loading the AAPCs with a pool of overlapping peptides derived from one or more WT1 peptides. In specific embodiments, the step of sensitizing the allogeneic cells using AAPCs comprises engineering the AAPCs to express at least one immunogenic WT1 peptide in the AAPCs.

In a specific embodiment, the pool of overlapping peptides is a pool of overlapping pentadecapeptides.

In various embodiments, the population of allogeneic cells is derived from a T cell line. In certain embodiments, the methods of treating WT1-positive multiple myeloma or plasma cell leukemia described herein further comprise, before the administering step, a step of selecting the T cell line from a bank of a plurality of cryopreserved T cell lines. In certain embodiments, the methods of treating WT1-positive multiple myeloma or plasma cell leukemia described herein further comprise, before the administering step, a step of thawing a cryopreserved form of the T cell line. In specific embodiments, the methods of treating WT1-positive multiple myeloma or plasma cell leukemia described herein further comprises, before the administering step, a step of expanding the T cell line in vitro.

In specific embodiments, the WT1-specific allogeneic T cells administered in accordance with the methods described herein recognize the RMFPNAPYL epitope of WT1.

In certain embodiments, the administering is by infusion of the population of allogeneic cells. In some embodiments, the infusion is bolus intravenous infusion. In certain embodiments, the administering comprises administering at least about 1×10⁵ cells of the population of allogeneic cells per kilogram per dose to the human patient. In some embodiments, the administering comprises administering about 1×10⁶ to about 5×10⁶ cells of the population of allogeneic cells per kilogram per dose to the human patient. In a specific embodiment, the administering comprises administering about 1×10⁶ cells of the population of allogeneic cells per kilogram per dose to the human patient. In another specific embodiment, the administering comprises administering about 3×10⁶ cells of the population of allogeneic cells per kilogram per dose to the human patient. In another specific embodiment, the administering comprises administering about 5×10⁶ cells of the population of allogeneic cells per kilogram per dose to the human patient.

In certain embodiments, the methods of treating WT1-positive multiple myeloma and plasma cell leukemia described herein comprise administering at least 2 doses of the population of allogeneic cells to the human patient. In specific embodiments, the methods of treating WT1-positive multiple myeloma and plasma cell leukemia described herein comprise administering 2, 3, 4, 5, or 6 doses of the population of allogeneic cells to the human patient. In a specific embodiment, the methods of treating WT1-positive multiple myeloma and plasma cell leukemia described herein comprise administering 3 doses of the population of allogeneic cells to the human patient.

In certain embodiments, the methods of treating WT1-positive multiple myeloma and plasma cell leukemia described herein comprise a washout period between two consecutive doses, wherein no dose of the population of allogeneic cells is administered during the washout period. In specific embodiments, the washout period is about 1, 2, 3, or 4 weeks. In a specific embodiment, the washout period is about 4 weeks.

In a specific embodiment, the administering comprises administering 3 doses to the human patient, each dose being in the range of 1×10⁶ to 5×10⁶ cells of the population of allogeneic cells per kilogram, and wherein the 3 doses are administered about 4 weeks apart from one another. In another specific embodiment, the administering comprises administering 3 doses to the human patient, each dose being in the range of 1×10⁶ to 5×10⁶ cells of the population of allogeneic cells per kilogram, and wherein the 3 doses are administered about 3 weeks apart from one another. In another specific embodiment, the administering comprises administering 3 doses to the human patient, each dose being in the range of 1×10⁶ to 5×10⁶ cells of the population of allogeneic cells per kilogram, and wherein the 2 doses are administered about 3 weeks apart from one another. In another specific embodiment, the administering comprises administering 3 doses to the human patient, each dose being in the range of 1×10⁶ to 5×10⁶ cells of the population of allogeneic cells per kilogram, and wherein the 3 doses are administered about 1 week apart from one another.

Also provided herein are methods of treating WT1-positive multiple myeloma or plasma cell leukemia which further comprise, after administering to the human patient the population of allogeneic cells, administering to the human patient a second population of allogeneic cells comprising WT1-specific allogeneic T cells; wherein the second population of allogeneic cells is restricted by a different HLA allele shared with the human patient.

### 4. BRIEF DESCRIPTION OF FIGURES

FIG. 1. WT1-specific T cell responses and disease evaluation following adoptive transfer of donor-derived WT1-specific T cells in patient with secondary plasma cell leukemia. (A) M-spike and (B) kappa: lambda ratio as disease marker post TCD HSCT is shown. Absolute numbers of CD3+CD8+ and of CD3+CD4+ following adoptive transfer of WT1-specific T cells. Frequencies of CD4+ and CD8+WT1-specific T cells in the peripheral blood of the patient were quantified by intracellular IFN-γ assay and shown at the individual time points. The patient achieved a CR following 2 cycles, each consisting of 3 infusions at 4 weekly intervals, of donor-derived WT1-specific CTLs.

FIG. 2. WT1-specific T cell responses and disease evaluation following adoptive transfer of donor-derived WT1-specific T cells in patient with primary plasma cell leukemia. Free kappa light chain as disease marker post TCD HSCT is shown. Absolute numbers of CD3+CD8+ and of CD3+CD4+ following adoptive transfer of WT1-specific T cells. Frequencies of CD4+ and CD8+WT1-specific T cells in the peripheral blood of the patient were quantified by intracellular IFN-γ assay and shown at the individual time points. The patient achieved a CR following 1 cycle, consisting of 3 infusions at 4 weekly intervals, of donor-derived WT1-specific CTLs.

FIG. 3. Cytogenetics measured in the enriched plasma cell population from bone marrow.

FIG. 4. Whole body tumor burden for H929 orthometastatic model mice treated with third-party T cell line from ATA 520 (** indicates p<0.01 by ANOVA for both Low and High Dose groups compared to Vehicle). Group means and distribution of individual disease burden are shown in FIG. 5 for H929 diseased animals on last day, day 28, post dose.

FIG. 5. Day 28 tumor burden, as both mean and individual values, for H929 diseased mice treated with T cell lines from ATA 520 (** indicates p<0.01 by one way ANOVA with correction compared to Vehicle; *** indicates p<0.001 across all groups by one way ANOVA; ns=not statistically significant by ANOVA with correction).

FIG. 6. Day 21 tumor burden, as both mean and individual values, for L363 model mice treated with T cell line from ATA 520 (* indicates p<0.05 by one way ANOVA with correction compared to Vehicle; ** indicates p<0.01 by one way ANOVA with correction compared to Vehicle; ns=not statistically significant by ANOVA with correction).

### 5. DETAILED DESCRIPTION

The present invention relates to methods of treating WT1 (Wilms Tumor 1)-positive multiple myeloma in a human patient. The present invention further relates to methods of treating WT1-positive plasma cell leukemia in a human patient. The invention provides a T cell therapy method that is effective in treating WT1-positive multiple myeloma and WT1-positive plasma cell leukemia in a human patient with low or no toxicity.

5.1. Methods of Treating Multiple Myeloma

Provided herein are methods of treating WT1-positive multiple myeloma in a human patient in need thereof, comprising administering to the human patient a population of allogeneic cells comprising WT1-specific allogeneic T cells.

In one aspect, the methods comprise administering to the human patient a population of allogeneic cells comprising WT1-specific allogeneic T cells, wherein the population of allogeneic cells lacks substantial cytotoxicity in vitro toward antigen presenting cells that are not WT1 peptide-loaded or genetically engineered to (i.e., recombinantly) express one or more WT1 peptides. Thus, the population of allogeneic cells does not have significant levels of alloreactivity, resulting generally in the absence of graft-versus-host disease (GvHD) in the human patient. In specific embodiments, the population of allogeneic cells lyses less than or equal to 15%, 10%, 5%, or 1% of antigen presenting cells that are not WT1 peptide-loaded or genetically engineered to (i.e., recombinantly) express one or more WT1 peptides. In a specific embodiment, the population of allogeneic cells lyses less than or equal to 15% of antigen presenting cells that are not WT1 peptide-loaded or genetically engineered to (i.e., recombinantly) express one or more WT1 peptides. In some embodiments, the antigen presenting cells are derived from the human patient, for example, unmodified phytohemagglutinin-stimulated lymphoblasts (i.e., phytohemagglutinin-stimulated lymphoblasts that are not loaded with one or more WT1 peptides and are not genetically engineered to express one or more WT1 peptides) derived from the human patient. In other embodiments, the antigen presenting cells are derived from the donor of the population of allogeneic cells, for example, unmodified phytohemagglutinin-stimulated lymphoblasts (i.e., phytohemagglutinin-stimulated lymphoblasts that are not loaded with one or more WT1 peptides and are not genetically engineered to express one or more WT1 peptides) derived from the donor of the population of allogeneic cells. In other embodiments, the antigen presenting cells are derived from unmodified HLA-mismatched cells of an Epstein Barr Virus-transformed B lymphocyte cell line (EBV BLCL) (i.e., cells of an EBV BLCL that are not loaded with one or more WT1 peptides and are not genetically engineered to express one or more WT1 peptides, and are HLA-mismatched relative to the population of allogeneic cells).

In a specific embodiment, the population of allogeneic cells lyses less than or equal to 15% of unmodified phytohemagglutinin-stimulated lymphoblasts derived from the human patient in an in vitro cytotoxicity assay, thereby lacking substantial cytotoxicity in vitro toward antigen presenting cells that are not WT peptide-loaded or genetically engineered to express one or more WT1 peptides.

In another specific embodiment, the population of allogeneic cells lyses less than or equal to 15% of unmodified phytohemagglutinin-stimulated lymphoblasts derived from the donor of the population of allogeneic cells in an in vitro cytotoxicity assay, thereby lacking substantial cytotoxicity in vitro toward antigen presenting cells that are not WT peptide-loaded or genetically engineered to express one or more WT1 peptides.

In another specific embodiment, the population of allogeneic cells lyses less than or equal to 15% of unmodified HLA-mismatched cells of an EBV BLCL in an in vitro cytotoxicity assay, thereby lacking substantial cytotoxicity in vitro toward antigen presenting cells that are not WT peptide-loaded or genetically engineered to express one or more WT1 peptides.

In another specific embodiment, the population of allogeneic cells lyses less than or equal to 15% of unmodified phytohemagglutinin-stimulated lymphoblasts derived from the human patient in an in vitro cytotoxicity assay, and the population of allogeneic cells lyses less than or equal to 15% of unmodified HLA-mismatched cells of the EBV BLCL in an in vitro cytotoxicity assay, thereby lacking substantial cytotoxicity in vitro toward antigen presenting cells that are not WT peptide-loaded or genetically engineered to express one or more WT1 peptides.

In another specific embodiment, the population of allogeneic cells lyses less than or equal to 15% of unmodified phytohemagglutinin-stimulated lymphoblasts derived from the donor of the population of allogeneic cells in an in vitro cytotoxicity assay, and the population of allogeneic cells lyses less than or equal to 15% of unmodified HLA-mismatched cells of the EBV BLCL in an in vitro cytotoxicity assay, thereby lacking substantial cytotoxicity in vitro toward antigen presenting cells that are not WT peptide-loaded or genetically engineered to express one or more WT1 peptides.

In a second aspect, the methods comprise administering to the human patient a population of allogeneic cells comprising WT1-specific allogeneic T cells, wherein the population of allogeneic cells exhibits substantial cytotoxicity in vitro toward (e.g., exhibits substantial lysis of) antigen presenting cells that are WT1 peptide-loaded or genetically engineered to (i.e., recombinantly) express one or more WT1 peptides. In specific embodiments, the population of allogeneic cells exhibits lysis of greater than or equal to 20%, 25%, 30%, 35%, or 40% of antigen presenting cells that are WT1 peptide-loaded in an in vitro cytotoxicity assay. In a specific embodiment, the population of allogeneic cells exhibits lysis of greater than or equal to 20% of antigen presenting cells that are WT1 peptide-loaded in an in vitro cytotoxicity assay. In some embodiments, the antigen presenting cells are derived from the human patient, for example, phytohemagglutinin-stimulated lymphoblasts derived from the human patient. In other embodiments, the antigen presenting cells are derived from the donor of the population of allogeneic cells, for example, phytohemagglutinin-stimulated lymphoblasts derived from the donor of the population of allogeneic cells.

In a specific embodiment, the population of allogeneic cells exhibits lysis of greater than or equal to 20% of WT1 peptide-loaded (e.g., WT1 peptide pool-loaded) phytohemagglutinin-stimulated lymphoblasts derived from the human patient in an in vitro cytotoxicity assay.

In another specific embodiment, the population of allogeneic cells exhibits lysis of greater than or equal to 20% of WT1 peptide-loaded (e.g., WT1 peptide pool-loaded) antigen presenting cells derived from the donor of the population of allogeneic cells in an in vitro cytotoxicity assay.

In another specific embodiment, the population of allogeneic cells exhibits lysis of greater than or equal to 20% of WT1 peptide-loaded (e.g., WT1 peptide pool-loaded) phytohemagglutinin-stimulated lymphoblasts derived from the human patient in an in vitro cytotoxicity assay, and exhibits lysis of greater than or equal to 20% of WT1 peptide-loaded (e.g., WT1 peptide pool-loaded) antigen presenting cells derived from the donor of the population of allogeneic cells in an in vitro cytotoxicity assay.

In specific embodiments, the antigen presenting cells are loaded with a pool of WT1 peptides. The pool of WT1 peptides, can be, for example, a pool of overlapping peptides (e.g., pentadecapeptides) spanning the sequence of WT1. In a specific embodiment, the pool of WT1 peptides is as described in the example of Section 6.

In a third aspect, the methods comprise administering to the human patient a population of allogeneic cells comprising WT1-specific allogeneic T cells, wherein the population of allogeneic cells lacks substantial cytotoxicity in vitro toward antigen presenting cells that are not WT1 peptide-loaded or genetically engineered to (i.e., recombinantly) express one or more WT1 peptides, as described above, and exhibits substantial cytotoxicity in vitro toward (e.g., exhibits substantial lysis of) antigen presenting cells that are WT1 peptide-loaded, as described above.

The cytotoxicity of a population of allogeneic cells toward antigen presenting cells can be determined by any assay known in the art to measure T cell mediated cytotoxicity. In a specific embodiment, the cytotoxicity is determined by a standard ⁵¹Cr release assay as described in the example of Section 6 or as described in Trivedi et al., 2005, Blood 105:2793-2801.

Antigen presenting cells that can be used in the in vitro cytotoxicity assay with the population of allogeneic cells include, but are not limited to, dendritic cells, phytohemagglutinin (PHA)-lymphoblasts, macrophages, B-cells that generate antibodies, cells of an EBV BLCL, and artificial antigen presenting cells (AAPCs).

In specific embodiment, the first dose of the population of allogeneic cells is administered within 12 weeks after the diagnosis of the multiple myeloma. In a specific embodiment, the first dose of the population of allogeneic cells is administered between 5 to 12 weeks after the diagnosis of the multiple myeloma. In another specific embodiment, the first dose of the population of allogeneic cells is administered between 6 to 12 weeks after the diagnosis of the multiple myeloma. In another specific embodiment, the first dose of the population of allogeneic cells is administered between 6 to 10 weeks after the diagnosis of the multiple myeloma. In another specific embodiment, the first dose of the population of allogeneic cells is administered between 6 to 8 weeks after the diagnosis of the multiple myeloma.

In various embodiments, prior to the administering of the population of allogeneic cells, the human patient has been administered a therapy for multiple myeloma that is different from said population of allogeneic cells. The therapy can be an autologous hematopoietic stem cell transplantation (HSCT), an allogeneic HSCT, a cancer chemotherapy, an induction therapy, a radiation therapy, or a combination thereof, to treat the multiple myeloma. When induction therapy is administered, it is often the first phase of treatment for multiple myeloma, and the goal is to reduce the number of plasma cells in the bone marrow and the proteins that the plasma cells produce. The induction therapy can be any induction therapy known in the art for treating multiple myeloma, and can be, for example, a chemotherapy, a targeted therapy, a treatment with corticosteroids, or a combination thereof. The autologous HSCT and/or the allogeneic HSCT can be a bone marrow transplant, a cord blood transplant, or preferably a peripheral blood stem cell transplant. The population of allogeneic cells can be derived from the donor of the allogeneic HSCT or a third-party donor that is different from the donor of the allogeneic HSCT. The cancer chemotherapy can be any chemotherapy known in the art for treating multiple myeloma. The radiation therapy can also be any radiation therapy known in the art for treating multiple myeloma. In certain embodiments, the first dose of the population of allogeneic cells is administered on the day of, or up to 12 weeks after, the ending of the last such therapy. In a specific embodiment, the first dose of the population of allogeneic cells is administered between 5 to 12 weeks after the ending of the last such therapy. In another specific embodiment, the first dose of the population of allogeneic cells is administered between 6 to 12 weeks after the ending of the last such therapy. In another specific embodiment, the first dose of the population of allogeneic cells is administered between 6 to 10 weeks after the ending of the last such therapy. In another specific embodiment, the first dose of the population of allogeneic cells is administered between 6 to 8 weeks after the ending of the last such therapy. In some specific embodiments, the last such therapy is an autologous HSCT. In other specific embodiments, the last such therapy is an allogeneic HSCT. For example, the last such therapy is an allogeneic HSCT that is administered after autologous HSCT, which is administered after induction therapy (e.g., induction chemotherapy).

In certain embodiments, the therapy is an HSCT. In certain embodiments, the therapy comprises an HSCT.

In specific embodiment, the therapy is an autologous HSCT. In specific embodiment, the therapy comprises an autologous HSCT. The autologous HSCT can be a peripheral blood stem cell transplant, a bone marrow transplant and cord blood transplant. In a specific embodiment, the autologous HSCT is a peripheral blood stem cell transplant. In some embodiments, the first dose of the population of allogeneic cells is administered on the day of, or up to 12 weeks after, the autologous HSCT. In a specific embodiment, the first dose of the population of allogeneic cells is administered between 5 to 12 weeks after the autologous HSCT. In another specific embodiment, the first dose of the population of allogeneic cells is administered between 6 to 12 weeks after the autologous HSCT. In another specific embodiment, the first dose of the population of allogeneic cells is administered between 6 to 10 weeks after the autologous HSCT. In another specific embodiment, the first dose of the population of allogeneic cells is administered between 6 to 8 weeks after the autologous HSCT.

In other specific embodiment, the therapy is an allogeneic HSCT (for example, a T cell-depleted allogeneic HSCT). In other specific embodiment, the therapy comprises an allogeneic HSCT (for example, a T cell-depleted allogeneic HSCT). The allogeneic HSCT can be a peripheral blood stem cell transplant, a bone marrow transplant and cord blood transplant. In a specific embodiment, the allogeneic HSCT is a peripheral blood stem cell transplant. The population of allogeneic cells can be derived from the donor of the allogeneic HSCT or a third-party donor that is different from the donor of the allogeneic HSCT. In some embodiments, the first dose of the population of allogeneic cells is administered on the day of, or up to 12 weeks after, the allogeneic HSCT. In a specific embodiment, the first dose of the population of allogeneic cells is administered between 5 to 12 weeks after the allogeneic HSCT. In another specific embodiment, the first dose of the population of allogeneic cells is administered between 6 to 12 weeks after the allogeneic HSCT. In another specific embodiment, the first dose of the population of allogeneic cells is administered between 6 to 10 weeks after the allogeneic HSCT. In another specific embodiment, the first dose of the population of allogeneic cells is administered between 6 to 8 weeks after the allogeneic HSCT.

In various embodiments, the human patient has failed the therapy prior to said administering of the population of allogeneic cells. A human patient is considered to have failed a therapy for multiple myeloma if the multiple myeloma is refractory to the therapy, relapses after the therapy, and/or if the human patient has discontinued the therapy due to intolerance of the therapy (for example, due to toxicity of the therapy in view of the patient's age or condition). If the therapy is or comprises allogeneic HSCT, the intolerance can be due to graft-versus-host disease (GvHD) caused by the allogeneic HSCT. In specific embodiments, the multiple myeloma is relapsed/refractory multiple myeloma (RRMM), which can be, for example, primary refractory multiple myeloma, relapsed multiple myeloma, or relapsed and refractory multiple myeloma. In a specific embodiment, the multiple myeloma is primary refractory multiple myeloma. In another specific embodiment, the multiple myeloma is relapsed multiple myeloma. In another specific embodiment, the multiple myeloma is relapsed and refractory multiple myeloma. Relapsed and refractory multiple myeloma is defined as progression of disease while on therapy in patients who achieve minor response (MR) or better, or who progress within 60 days of their last therapy. Patients who never achieve at least a MR to initial induction therapy and progress while on therapy are defined as “primary refractory.” Relapsed multiple myeloma is defined as disease in a myeloma patient who has previously been treated and has achieved remission, and has evidence of PD (progressive disease) as defined below, and who at the time of relapse does not meet the criteria for relapsed and refractory or primary refractory multiple myeloma According to the International Myeloma Working Group criteria, PD is defined by at least a 25% increase from nadir in the serum paraprotein (absolute increase must be ≥0.5 g/dL) or urine paraprotein (absolute increase must be ≥200 mg/24 hours), or in the difference between involved and uninvolved serum-free light-chain (FLC) levels (with an abnormal FLC ratio and FLC difference >100 mg/L). In patients who lack measurable paraprotein levels (oligo- or nonsecretory myeloma), an increase in bone marrow plasma cells (≥10% increase) or new bone/soft tissue lesions increasing the size of existing lesions or unexplained serum calcium >11.5 mg/dL is used to define PD. In a specific embodiment, the human patient has failed a combination chemotherapy (e.g., a combination chemotherapy comprising treatment with lenalidomide and bortezomib). In a specific embodiment, the human patient has failed multiple lines of treatment including a combination chemotherapy (e.g., a combination chemotherapy comprising treatment with lenalidomide and bortezomib) and an autologous HSCT.

In other various embodiments, prior to the administering of the population of allogeneic cells, the human patient has not been administered a therapy for multiple myeloma. In such embodiments, the population of allogeneic cells is administered as a front-line therapy for multiple myeloma. In specific embodiment, the first dose of the population of allogeneic cells is administered within 12 weeks after the diagnosis of the multiple myeloma. In a specific embodiment, the first dose of the population of allogeneic cells is administered between 5 to 12 weeks after the diagnosis of the multiple myeloma. In another specific embodiment, the first dose of the population of allogeneic cells is administered between 6 to 12 weeks after the diagnosis of the multiple myeloma. In another specific embodiment, the first dose of the population of allogeneic cells is administered between 6 to 10 weeks after the diagnosis of the multiple myeloma. In another specific embodiment, the first dose of the population of allogeneic cells is administered between 6 to 8 weeks after the diagnosis of the multiple myeloma.

In specific embodiments of the methods of treating WT1-positive multiple myeloma as described above, the administering of the population of allogeneic cells does not result in any graft-versus-host disease (GvHD) in the human patient.

5.2. Methods of Treating Plasma Cell Leukemia

Also provided herein are methods of treating WT1-positive plasma cell leukemia in a human patient in need thereof, comprising administering to the human patient a population of allogeneic cells comprising WT1-specific allogeneic T cells.

In one aspect, the methods comprise administering to the human patient a population of allogeneic cells comprising WT1-specific allogeneic T cells, wherein the population of allogeneic cells lacks substantial cytotoxicity in vitro toward antigen presenting cells that are not WT1 peptide-loaded or genetically engineered to (i.e., recombinantly) express one or more WT1 peptides. Thus, the population of allogeneic cells does not have significant levels of alloreactivity, resulting generally in the absence of graft-versus-host disease (GvHD) in the human patient. In specific embodiments, the population of allogeneic cells lyses less than or equal to 15%, 10%, 5%, or 1% of antigen presenting cells that are not WT1 peptide-loaded or genetically engineered to (i.e., recombinantly) express one or more WT1 peptides. In a specific embodiment, the population of allogeneic cells lyses less than or equal to 15% of antigen presenting cells that are not WT1 peptide-loaded or genetically engineered to (i.e., recombinantly) express one or more WT1 peptides. In some embodiments, the antigen presenting cells are derived from the human patient, for example, unmodified phytohemagglutinin-stimulated lymphoblasts (i.e., phytohemagglutinin-stimulated lymphoblasts that are not loaded with one or more WT1 peptides and are not genetically engineered to express one or more WT1 peptides) derived from the human patient. In other embodiments, the antigen presenting cells are derived from the donor of the population of allogeneic cells, for example, unmodified phytohemagglutinin-stimulated lymphoblasts (i.e., phytohemagglutinin-stimulated lymphoblasts that are not loaded with one or more WT1 peptides and are not genetically engineered to express one or more WT1 peptides) derived from the donor of the population of allogeneic cells. In other embodiments, the antigen presenting cells are derived from unmodified HLA-mismatched cells of an Epstein Barr Virus-transformed B lymphocyte cell line (EBV BLCL) (i.e., cells of an EBV BLCL that are not loaded with one or more WT1 peptides and are not genetically engineered to express one or more WT1 peptides, and are HLA-mismatched relative to the population of allogeneic cells).

In a specific embodiment, the population of allogeneic cells lyses less than or equal to 15% of unmodified phytohemagglutinin-stimulated lymphoblasts derived from the human patient in an in vitro cytotoxicity assay, thereby lacking substantial cytotoxicity in vitro toward antigen presenting cells that are not WT peptide-loaded or genetically engineered to express one or more WT1 peptides.

In another specific embodiment, the population of allogeneic cells lyses less than or equal to 15% of unmodified phytohemagglutinin-stimulated lymphoblasts derived from the donor of the population of allogeneic cells in an in vitro cytotoxicity assay, thereby lacking substantial cytotoxicity in vitro toward antigen presenting cells that are not WT peptide-loaded or genetically engineered to express one or more WT1 peptides.

In another specific embodiment, the population of allogeneic cells lyses less than or equal to 15% of unmodified HLA-mismatched cells of an EBV BLCL in an in vitro cytotoxicity assay, thereby lacking substantial cytotoxicity in vitro toward antigen presenting cells that are not WT peptide-loaded or genetically engineered to express one or more WT1 peptides.

In another specific embodiment, the population of allogeneic cells lyses less than or equal to 15% of unmodified phytohemagglutinin-stimulated lymphoblasts derived from the human patient in an in vitro cytotoxicity assay, and the population of allogeneic cells lyses less than or equal to 15% of unmodified HLA-mismatched cells of the EBV BLCL in an in vitro cytotoxicity assay, thereby lacking substantial cytotoxicity in vitro toward antigen presenting cells that are not WT peptide-loaded or genetically engineered to express one or more WT1 peptides.

In another specific embodiment, the population of allogeneic cells lyses less than or equal to 15% of unmodified phytohemagglutinin-stimulated lymphoblasts derived from the donor of the population of allogeneic cells in an in vitro cytotoxicity assay, and the population of allogeneic cells lyses less than or equal to 15% of unmodified HLA-mismatched cells of the EBV BLCL in an in vitro cytotoxicity assay, thereby lacking substantial cytotoxicity in vitro toward antigen presenting cells that are not WT peptide-loaded or genetically engineered to express one or more WT1 peptides.

In a second aspect, the methods comprise administering to the human patient a population of allogeneic cells comprising WT1-specific allogeneic T cells, wherein the population of allogeneic cells exhibits substantial cytotoxicity in vitro toward (e.g., exhibits substantial lysis of) antigen presenting cells that are WT1 peptide-loaded. In specific embodiments, the population of allogeneic cells exhibits lysis of greater than or equal to 20%, 25%, 30%, 35%, or 40% of antigen presenting cells that are WT1 peptide-loaded in an in vitro cytotoxicity assay. In a specific embodiment, the population of allogeneic cells exhibits lysis of greater than or equal to 20% of antigen presenting cells that are WT1 peptide-loaded in an in vitro cytotoxicity assay. In some embodiments, the antigen presenting cells are derived from the human patient, for example, phytohemagglutinin-stimulated lymphoblasts derived from the human patient. In other embodiments, the antigen presenting cells are derived from the donor of the population of allogeneic cells, for example, phytohemagglutinin-stimulated lymphoblasts derived from the donor of the population of allogeneic cells.

In a specific embodiment, the population of allogeneic cells exhibits lysis of greater than or equal to 20% of WT1 peptide-loaded (WT1 peptide pool-loaded) phytohemagglutinin-stimulated lymphoblasts derived from the human patient in an in vitro cytotoxicity assay.

In another specific embodiment, the population of allogeneic cells exhibits lysis of greater than or equal to 20% of WT1 peptide-loaded (e.g., WT1 peptide pool-loaded) antigen presenting cells derived from the donor of the population of allogeneic cells in an in vitro cytotoxicity assay.

In another specific embodiment, the population of allogeneic cells exhibits lysis of greater than or equal to 20% of WT1 peptide-loaded (e.g., WT1 peptide pool-loaded) phytohemagglutinin-stimulated lymphoblasts derived from the human patient in an in vitro cytotoxicity assay, and exhibits lysis of greater than or equal to 20% of WT1 peptide-loaded (e.g., WT1 peptide pool-loaded) antigen presenting cells derived from the donor of the population of allogeneic cells in an in vitro cytotoxicity assay.

In specific embodiments, the antigen presenting cells are loaded with a pool of WT1 peptides. The pool of WT1 peptides, can be, for example, a pool of overlapping peptides (e.g., pentadecapeptides) spanning the sequence of WT1. In a specific embodiment, the pool of WT1 peptides is as described in the example of Section 6.

In a third aspect, the methods comprise administering to the human patient a population of allogeneic cells comprising WT1-specific allogeneic T cells, wherein the population of allogeneic cells lacks substantial cytotoxicity in vitro toward antigen presenting cells that are not WT1 peptide-loaded or genetically engineered to (i.e., recombinantly) express one or more WT1 peptides, as described above, and exhibits substantial cytotoxicity in vitro toward (e.g., exhibits substantial lysis of) antigen presenting cells that are WT1 peptide-loaded, as described above.

The cytotoxicity of a population of allogeneic cells toward antigen presenting cells can be determined by any assay known in the art to measure T cell mediated cytotoxicity. In a specific embodiment, the cytotoxicity is determined by a standard ⁵¹Cr release assay as described in the example of Section 6 or as described in Trivedi et al., 2005, Blood 105:2793-2801.

Antigen presenting cells that can be used in the in vitro cytotoxicity assay with the population of allogeneic cells include, but are not limited to, dendritic cells, phytohemagglutinin (PHA)-lymphoblasts, macrophages, B-cells that generate antibodies, and artificial antigen presenting cells (AAPCs).

In some embodiments, the plasma cell leukemia is primary plasma cell leukemia. In other embodiments, the plasma cell leukemia is secondary plasma cell leukemia. Primary plasma cell leukemia is defined by the presence of >2×10⁹/L peripheral blood plasma cells or plasmacytosis accounting for >20% of the differential white cell count, and does not arise from pre-existing multiple myeloma (MM) (Jaffe et al., 2001, Ann Oncol 13:490-491; Hayman and Fonseca, 2001, Curr Treat Options Oncol 2:205-216). Secondary PCL (sPCL), however, is a leukemic transformation of end stage MM.

In specific embodiment, the first dose of the population of allogeneic cells is administered within 12 weeks after the diagnosis of the plasma cell leukemia. In a specific embodiment, the first dose of the population of allogeneic cells is administered between 5 to 12 weeks after the diagnosis of the plasma cell leukemia. In another specific embodiment, the first dose of the population of allogeneic cells is administered between 6 to 12 weeks after the diagnosis of the plasma cell leukemia. In another specific embodiment, the first dose of the population of allogeneic cells is administered between 6 to 10 weeks after the diagnosis of the plasma cell leukemia. In another specific embodiment, the first dose of the population of allogeneic cells is administered between 6 to 8 weeks after the diagnosis of the plasma cell leukemia.

In various embodiments, prior to the administering of the population of allogeneic cells, the human patient has been administered a therapy for plasma cell leukemia that is different from said population of allogeneic cells. The therapy can be an autologous hematopoietic stem cell transplantation (HSCT), an allogeneic HSCT, a cancer chemotherapy, an induction therapy, a radiation therapy, or a combination thereof, to treat the plasma cell leukemia. When induction therapy is administered, it is often the first phase of treatment for plasma cell leukemia, and the goal is to reduce the number of plasma cells in the bone marrow and the proteins that the plasma cells produce. The induction therapy can be any induction therapy known in the art for treating plasma cell leukemia, and can be, for example, a chemotherapy, a targeted therapy, a treatment with corticosteroids, or a combination thereof. The autologous HSCT and/or the allogeneic HSCT can be a bone marrow transplant, a cord blood transplant, or preferably a peripheral blood stem cell transplant. The population of allogeneic cells can be derived from the donor of the allogeneic HSCT or a third-party donor that is different from the donor of the allogeneic HSCT. The cancer chemotherapy can be any chemotherapy known in the art for treating plasma cell leukemia. The radiation therapy can also be any radiation therapy known in the art for treating plasma cell leukemia. In certain embodiments, the first dose of the population of allogeneic cells is administered on the day of, or up to 12 weeks after, the ending of the last such therapy. In a specific embodiment, the first dose of the population of allogeneic cells is administered between 5 to 12 weeks after the ending of the last such therapy. In another specific embodiment, the first dose of the population of allogeneic cells is administered between 6 to 12 weeks after the ending of the last such therapy. In another specific embodiment, the first dose of the population of allogeneic cells is administered between 6 to 10 weeks after the ending of the last such therapy. In another specific embodiment, the first dose of the population of allogeneic cells is administered between 6 to 8 weeks after the ending of the last such therapy. In some specific embodiments, the last such therapy is an autologous HSCT. In other specific embodiments, the last such therapy is an allogeneic HSCT. For example, the last such therapy is an allogeneic HSCT that is administered after autologous HSCT, which is administered after induction therapy (e.g., induction chemotherapy).

In certain embodiments, the therapy is an HSCT. In certain embodiments, the therapy comprises an HSCT.

In specific embodiment, the therapy is an autologous HSCT. In specific embodiment, the therapy comprises an autologous HSCT. The autologous HSCT can be a peripheral blood stem cell transplant, a bone marrow transplant and cord blood transplant. In a specific embodiment, the autologous HSCT is a peripheral blood stem cell transplant. In some embodiments, the first dose of the population of allogeneic cells is administered on the day of, or up to 12 weeks after, the autologous HSCT. In a specific embodiment, the first dose of the population of allogeneic cells is administered between 5 to 12 weeks after the autologous HSCT. In another specific embodiment, the first dose of the population of allogeneic cells is administered between 6 to 12 weeks after the autologous HSCT. In another specific embodiment, the first dose of the population of allogeneic cells is administered between 6 to 10 weeks after the autologous HSCT. In another specific embodiment, the first dose of the population of allogeneic cells is administered between 6 to 8 weeks after the autologous HSCT.

In other specific embodiment, the therapy is an allogeneic HSCT (for example, a T cell-depleted allogeneic HSCT). In other specific embodiment, the therapy comprises an allogeneic HSCT (for example, a T cell-depleted allogeneic HSCT). The allogeneic HSCT can be a peripheral blood stem cell transplant, a bone marrow transplant and cord blood transplant. In a specific embodiment, the allogeneic HSCT is a peripheral blood stem cell transplant. The population of allogeneic cells can be derived from the donor of the allogeneic HSCT or a third-party donor that is different from the donor of the allogeneic HSCT. In some embodiments, the first dose of the population of allogeneic cells is administered on the day of, or up to 12 weeks after, the allogeneic HSCT. In a specific embodiment, the first dose of the population of allogeneic cells is administered between 5 to 12 weeks after the allogeneic HSCT. In another specific embodiment, the first dose of the population of allogeneic cells is administered between 6 to 12 weeks after the allogeneic HSCT. In another specific embodiment, the first dose of the population of allogeneic cells is administered between 6 to 10 weeks after the allogeneic HSCT. In another specific embodiment, the first dose of the population of allogeneic cells is administered between 6 to 8 weeks after the allogeneic HSCT.

In various embodiments, the human patient has failed the therapy prior to said administering of the population of allogeneic cells. A human patient is considered to have failed a therapy for plasma cell leukemia if the plasma cell leukemia is refractory to the therapy, relapses after the therapy, and/or if the human patient has discontinued the therapy due to intolerance of the therapy (for example, due to toxicity of the therapy in view of the patient's age or condition). If the therapy is or comprises allogeneic HSCT, the intolerance can be due to graft-versus-host disease (GvHD) caused by the allogeneic HSCT. Since plasma cell leukemia is such an aggressive disease with short progression free-survivals, almost all patients are refractory. A plasma cell leukemia is considered refractory to a therapy, if the plasma cell leukemia has no response, or has residual disease, or progresses while on the therapy. In a specific embodiment, the human patient has failed a combination chemotherapy (e.g., VDT-PACE, RVD, or a combination thereof). VDT-PACE is a combination chemotherapy regimen with bortezomib, dexamethasone, thalidomide, cisplatin, doxorubicin, cyclophosphamide, and etoposide. RVD is a combination chemotherapy regimen with lenalidomide, bortezomib, and dexamethasone. In a specific embodiment, the human patient has failed multiple lines of treatment including a combination chemotherapy (e.g., VDT-PACE, RVD, or a combination thereof) and an autologous HSCT.

In other various embodiments, prior to the administering of the population of allogeneic cells, the human patient has not been administered a therapy for plasma cell leukemia. In such embodiments, the population of allogeneic cells is administered as a front-line therapy for plasma cell leukemia. In specific embodiment, the first dose of the population of allogeneic cells is administered within 12 weeks after the diagnosis of the plasma cell leukemia. In a specific embodiment, the first dose of the population of allogeneic cells is administered between 5 to 12 weeks after the diagnosis of the plasma cell leukemia. In another specific embodiment, the first dose of the population of allogeneic cells is administered between 6 to 12 weeks after the diagnosis of the plasma cell leukemia. In another specific embodiment, the first dose of the population of allogeneic cells is administered between 6 to 10 weeks after the diagnosis of the plasma cell leukemia. In another specific embodiment, the first dose of the population of allogeneic cells is administered between 6 to 8 weeks after the diagnosis of the plasma cell leukemia.

In specific embodiments of the methods of treating WT1-positive plasma cell leukemia as described above, the administering of the population of allogeneic cells does not result in any graft-versus-host disease (GvHD) in the human patient.

5.3. A Population of Allogeneic Cells Restricted by an Shared HLA Allele with the Human Patient

According to the invention, a population of allogeneic cells comprising WT1-specific allogeneic T cells is administered to the human patient. In a specific embodiment, the population of allogeneic cells that is administered to the human patient is restricted by an HLA allele shared with the human patient. This HLA allele restriction can be ensured by ascertaining the HLA assignment of the human patient (e.g., by using cells or tissue from the human patient), and selecting a population of allogeneic cells comprising WT1-specific allogeneic T cells (or a T cell line from which to derive the population of allogeneic cells) restricted by an HLA allele of the human patient.

In some embodiments of ascertaining an HLA assignment, at least 4 HLA loci (preferably HLA-A, HLA-B, HLA-C, and HLA-DR) are typed. In some embodiments of ascertaining an HLA assignment, 4 HLA loci (preferably HLA-A, HLA-B, HLA-C, and HLA-DR) are typed. In some embodiments of ascertaining an HLA assignment, 6 HLA loci are typed. In some embodiments of ascertaining an HLA assignment, 8 HLA loci are typed.

In certain embodiments, preferably in addition to being restricted by an HLA allele shared with the human patient, the population of allogeneic cells comprising WT1-specific allogeneic T cells shares at least 2 HLA alleles with the human patient. In specific embodiments, the population of allogeneic cells comprising WT1-specific allogeneic T cells shares at least 2 out of 8 HLA alleles (for example, two HLA-A alleles, two HLA-B alleles, two HLA-C alleles, and two HLA-DR alleles) with the human patient. This sharing can be ensured by ascertaining the HLA assignment of the human patient (e.g., by using cells or tissue from the human patient), and selecting a population of allogeneic cells comprising WT1-specific allogeneic T cells (or a T cell line from which to derive the population of allogeneic cells) that shares at least 2 (e.g., at least 2 out of 8) HLA alleles with the human patient.

The HLA assignment (i.e., the HLA loci type) can be ascertained (i.e., typed) by any method known in the art. Non-limiting exemplary methods for ascertaining the HLA assignment can be found in ASHI Laboratory Manual, Edition 4.2 (2003), American Society for Histocompatibility and Immunogenetics; ASHI Laboratory Manual, Supplements 1 (2006) and 2 (2007), American Society for Histocompatibility and Immunogenetics; Hurley, “DNA-based typing of HLA for transplantation.” in Leffell et al., eds., 1997, Handbook of Human Immunology, Boca Raton: CRC Press; Dunn, 2011, Int J Immunogenet 38:463-473; Erlich, 2012, Tissue Antigens, 80:1-11; Bontadini, 2012, Methods, 56:471-476; and Lange et al., 2014, BMC Genomics 15: 63.

In general, high-resolution typing is preferable for HLA typing. The high-resolution typing can be performed by any method known in the art, for example, as described in ASHI Laboratory Manual, Edition 4.2 (2003), American Society for Histocompatibility and Immunogenetics; ASHI Laboratory Manual, Supplements 1 (2006) and 2 (2007), American Society for Histocompatibility and Immunogenetics; Flomenberg et al., Blood, 104:1923-1930; Kogler et al., 2005, Bone Marrow Transplant, 36:1033-1041; Lee et al., 2007, Blood 110:4576-4583; Erlich, 2012, Tissue Antigens, 80:1-11; Lank et al., 2012, BMC Genomics 13:378; or Gabriel et al., 2014, Tissue Antigens, 83:65-75. In specific embodiments, the methods of treating WT1-positive multiple myeloma or plasma cell leukemia described herein further comprise prior to the administering step a step of ascertaining at least one HLA allele of the human patient by high-resolution typing.

The HLA allele by which the population of allogeneic cells is restricted can be determined by any method known in the art, for example, as described in Trivedi et al., 2005, Blood 105:2793-2801; Barker et al., 2010, Blood 116:5045-5049; Hasan et al., 2009, J Immunol, 183:2837-2850; or Doubrovina et al., 2012, Blood 120:1633-1646.

Preferably, the HLA allele by which the population of allogeneic cells is restricted and is shared with the human patient is defined by high-resolution typing. Preferably, the HLA alleles that are shared between the population of allogeneic cells and the human patient are defined by high-resolution typing. Most preferably, both the HLA allele by which the population of allogeneic cells is restricted and is shared with the human patient, and the HLA alleles that are shared between the population of allogeneic cells and the human patient are defined by high-resolution typing.

5.4. Obtaining or Generating a Population of Allogeneic Cells Comprising WT1-Specific Allogeneic T Cells

The population of allogeneic cells comprising WT1-specific allogeneic T cells that is administered to the human patient can be generated by a method known in the art, or can be selected from a preexisting bank (collection) of cryopreserved T cell lines (each T cell line comprising WT1-specific allogeneic T cells) generated by a method known in the art, and thawed and preferably expanded prior to administration. Preferably, unique identifier for each T cell line in the bank is associated with information as to which HLA allele(s) the respective T cell line is restricted, the HLA assignment of the respective T cell line, and/or the anti-WT1 cytotoxic activity of the respective T cell line measured by a method known in the art (for example, as described in Trivedi et al., 2005, Blood 105:2793-2801; or Hasan et al., 2009, J Immunol 183: 2837-2850). The population of allogeneic cells and the T cell lines in the bank are preferably obtained or generated by methods described below.

In various embodiments, the methods of treating WT1-positive multiple myeloma or plasma cell leukemia further comprise prior to the administering step a step of obtaining the population of allogeneic cells.

In specific embodiments, the step of obtaining the population of allogeneic cells comprises fluorescence activated cell sorting for WT1-specific T cells from a population of blood cells. In a specific embodiment, the population of blood cells are peripheral blood mononuclear cells (PBMCs) isolated from a blood sample(s) obtained from a human donor. The fluorescence activated cell sorting can be performed by any method known in the art, which normally involves staining the population of blood cells with an antibody that recognizes at least one WT1 epitope before the sorting step.

In specific embodiments, the step of obtaining the population of allogeneic cells comprises generating the population of allogeneic cells in vitro. The population of allogeneic cells can be generated in vitro by any method known in the art. Non-limiting exemplary methods of generating the population of allogeneic cells can be found in Trivedi et al., 2005, Blood 105:2793-2801; Hasan et al., 2009, J Immunol 183: 2837-2850; Koehne et al., 2015, Biol Blood Marrow Transplant S1083-8791(15)00372-9, published online May 29, 2015; O'Reilly et al., 2007, Immunol Res 38:237-250; Doubrovina et al., 2012, Blood 120:1633-1646; and O'Reilly et al., 2011, Best Practice & Research Clinical Haematology 24:381-391.

In certain embodiments, the step of generating the population of allogeneic cells in vitro comprises sensitizing (i.e., stimulating) allogeneic cells (which comprise allogeneic T cells) to one or more WT1 peptides so as to produce WT1-specific allogeneic T cells. A WT1 peptide can be the full-length WT1 protein (e.g., the full-length human WT1 protein), or a fragment thereof (e.g., a pentadecapeptide fragment of WT1). In specific embodiments, the step of generating the population of allogeneic cells in vitro comprises sensitizing allogeneic cells to one or more WT1 peptides presented by antigen presenting cells. In a specific embodiment, the sensitizing is carried out by culturing the allogeneic cells with the antigen presenting cells over a time period sufficient for sensitization and to reduce alloreactivity. This can be carried out, by way of example, by culturing the allogeneic cells with the antigen presenting cells over 6 to 8 weeks of culture.

The allogeneic cells that are used for generating the population of allogeneic cells in vitro can be isolated from the donor of the allogeneic cells by any method known in the art, for example, as described in Trivedi et al., 2005, Blood 105:2793-2801; Hasan et al., 2009, J Immunol 183: 2837-2850; or O'Reilly et al., 2007, Immunol Res. 38:237-250.

In a specific embodiment, the step of generating the population of allogeneic cells in vitro comprises a step of enriching for T cells prior to said sensitizing. The T cells can be enriched, for example, from peripheral blood lymphocytes separated from PBMCs of the donor of the allogeneic cells. In a specific embodiment, T cells are enriched from peripheral blood lymphocytes separated from PBMCs of the donor of the allogeneic cells by depletion of adherent monocytes followed by depletion of natural killer cells. In a specific embodiment, the step of generating the population of allogeneic cells in vitro comprises a step of purifying T cells prior to said sensitizing. The T cells can be purified, for example, by contacting PBMCs with antibodies recognizing T cell-specific marker(s).

In various embodiments, the allogeneic cells are cryopreserved for storage. In a specific embodiment, wherein the allogeneic cells are cryopreserved, the cryopreserved allogeneic cells are thawed and expanded in vitro before sensitizing. In a specific embodiment, wherein the allogeneic cells are cryopreserved, the cryopreserved allogeneic cells are thawed and then sensitized, but not expanded in vitro before sensitizing, and then optionally expanded. In specific embodiments, the allogeneic cells are cryopreserved after sensitizing (sensitizing produces the WT1-specific allogeneic cells). In a specific embodiment, wherein the allogeneic cells are cryopreserved after sensitizing, the cryopreserved allogeneic cells are thawed and expanded in vitro to produce the population of allogeneic cells comprising WT1-specific allogeneic T cells. In another specific embodiment, wherein the allogeneic cells are cryopreserved after sensitizing, the cryopreserved allogeneic cells are thawed but not expanded in vitro to produce the population of allogeneic cells comprising WT1-specific allogeneic T cells. In other various embodiments, the allogeneic cells are not cryopreserved. In a specific embodiment, wherein the allogeneic cells are not cryopreserved, the allogeneic cells are expanded in vitro before sensitizing. In a specific embodiment, wherein the allogeneic cells are not cryopreserved, the allogeneic cells are not expanded in vitro before sensitizing. In specific embodiments, the step of generating the population of allogeneic cells in vitro further comprises, after sensitizing, cryopreserving the allogeneic cells.

In specific embodiments, the methods of treating WT1-positive multiple myeloma or plasma cell leukemia described herein further comprise, before the administering step, steps of thawing cryopreserved WT1-peptide sensitized allogeneic cells, and expanding the allogeneic cells in vitro, to produce the population of allogeneic cells.

In certain embodiments, the step of generating the population of allogeneic cells in vitro comprises sensitizing the allogeneic cells using dendritic cells (preferably, the dendritic cells are derived from the donor of the allogeneic cells). In specific embodiments, the step of sensitizing the allogeneic cells using dendritic cells comprises loading the dendritic cells with at least one immunogenic peptide derived from WT1. In specific embodiments, the step of sensitizing the allogeneic cells using dendritic cells comprises loading the dendritic cells with a pool of overlapping peptides derived from one or more WT1 peptides.

In certain embodiments, the step of generating the population of allogeneic cells in vitro comprises sensitizing the allogeneic T cells using cytokine-activated monocytes (preferably, the cytokine-activated monocytes are derived from the donor of the allogeneic cells). In specific embodiments, the step of sensitizing the allogeneic cells using cytokine-activated monocytes comprises loading the cytokine-activated monocytes with at least one immunogenic peptide derived from WT1. In specific embodiments, the step of sensitizing the allogeneic cells using cytokine-activated monocytes comprises loading the cytokine-activated monocytes with a pool of overlapping peptides derived from one or more WT1 peptides.

In certain embodiments, the step of generating the population of allogeneic cells in vitro comprises sensitizing the allogeneic cells using peripheral blood mononuclear cells (preferably, the peripheral blood mononuclear cells are derived from the donor of the allogeneic cells). In specific embodiments, the step of sensitizing the allogeneic cells using peripheral blood mononuclear cells comprises loading the peripheral blood mononuclear cells with at least one immunogenic peptide derived from WT1. In specific embodiments, the step of sensitizing the allogeneic cells using peripheral blood mononuclear cells comprises loading the peripheral blood mononuclear cells with a pool of overlapping peptides derived from one or more WT1 peptides.

In certain embodiments, the step of generating the population of allogeneic cells in vitro comprises sensitizing the allogeneic cells using EBV-transformed B lymphocyte cell line (EBV-BLCL) cells, for example, an EBV strain B95.8-transformed B lymphocyte cell line (preferably, the EBV-BLCL is derived from the donor of allogeneic T cells). The EBV-BLCL cells can be generated by any method known in the art, or as previously described in Trivedi et al., 2005, Blood 105:2793-2801 or Hasan et al., 2009, J Immunol 183:2837-2850. In specific embodiments, the step of sensitizing the allogeneic cells using EBV-BLCL cells comprises loading the EBV-BLCL cells with at least one immunogenic peptide derived from WT1. In specific embodiments, the step of sensitizing the allogeneic cells using EBV-BLCL cells comprises loading the EBV-BLCL cells with a pool of overlapping peptides derived from one or more WT1 peptides.

In certain embodiments, the step of generating the population of allogeneic cells in vitro comprises sensitizing the allogeneic cells using artificial antigen-presenting cells (AAPCs). In specific embodiments, the step of sensitizing the allogeneic T cells using AAPCs comprises loading the AAPCs with at least one immunogenic peptide derived from WT1. In specific embodiments, the step of sensitizing the allogeneic T cells using AAPCs comprises loading the AAPCs with a pool of overlapping peptides derived from one or more WT1 peptides. In specific embodiments, the step of sensitizing the allogeneic cells using AAPCs comprises engineering the AAPCs to express at least one immunogenic WT1 peptide in the AAPCs.

In various embodiments, the pool of peptides is a pool of overlapping peptides spanning WT1 (e.g., human WT1). In a specific embodiment, the pool of overlapping peptides is a pool of overlapping pentadecapeptides.

In specific embodiments, the population of allogeneic cells has been cryopreserved for storage before administering. In specific embodiments, the population of allogeneic cells has not been cryopreserved for storage before administering. In certain embodiments, the methods of treating WT1-positive multiple myeloma or plasma cell leukemia described herein further comprise, before the administering step, a step of thawing a cryopreserved form of the population of allogeneic cells.

In various embodiments, the population of allogeneic cells is derived from a T cell line. The T cell line contains T cells, but the percentage of T cells may be less than 100%, 90%, 80%, 70%, 60%, 50%, 40%, 30%, 20% or 10%. In specific embodiments, the T cell line has been cryopreserved for storage before administering. In specific embodiments, the T cell line has not been cryopreserved for storage before administering. In some embodiments, the T cell line has been expanded in vitro to derive the population of allogeneic cells. In other embodiments, the T cell line has not been expanded in vitro to derive the population of allogeneic cells. The T cell line can be sensitized to one or more WT1 peptides (so as to produce WT1-specific allogeneic T cells, for example, by a sensitizing step described above) before or after cryopreservation (if the T cell line has been cryopreserved), and before or after expanding in vitro (if the T cell line has been expanded in vitro). In certain embodiments, the methods of treating WT1-positive multiple myeloma or plasma cell leukemia described herein further comprise, before the administering step, a step of selecting the T cell line from a bank of a plurality of cryopreserved T cell lines (preferably each comprising WT1-specific allogeneic T cells). Preferably, unique identifier for each T cell line in the bank is associated with information as to which HLA allele(s) the respective T cell line is restricted, and optionally also information as to the HLA assignment of the respective T cell line. In certain embodiments, the methods of treating WT1-positive multiple myeloma or plasma cell leukemia described herein further comprise, before the administering step, a step of thawing a cryopreserved form of the T cell line. In specific embodiments, the methods of treating WT1-positive multiple myeloma or plasma cell leukemia described herein further comprises, before the administering step, a step of expanding the T cell line (for example, after thawing a cryopreserved form of the T cell line) in vitro. The T cell line and the plurality of cryopreserved T cell lines can be generated by any method known in the art, for example, as described in Trivedi et al., 2005, Blood 105:2793-2801; Hasan et al., 2009, J Immunol 183: 2837-2850; Koehne et al., 2015, Biol Blood Marrow Transplant S1083-8791(15)00372-9, published online May 29, 2015; O'Reilly et al., 2007, Immunol Res 38:237-250; or O'Reilly et al., 2011, Best Practice & Research Clinical Haematology 24:381-391, or as describe above for generating the population of allogeneic cells in vitro.

The population of allogeneic cells comprising WT1-specific allogeneic T cells that is administered to the human patient comprises CD8+ T cells, and in a specific embodiment also comprises CD4+ T cells.

The WT1-specific allogeneic T cells administered in accordance with the methods described herein recognize at least one epitope of WT1. In specific embodiments, the WT1-specific allogeneic T cells administered in accordance with the methods described herein recognize the RMFPNAPYL epitope of WT1. In a specific embodiment, the WT1-specific allogeneic T cells administered in accordance with the methods described herein recognize the RMFPNAPYL epitope presented by HLA-A0201.

5.5. Administration and Dosage

The route of administration of the population of allogeneic cells and the amount to be administered to the human patient can be determined based on the condition of the human patient and the knowledge of the physician. Generally, the administration is intravenous.

In certain embodiments, the administering is by infusion of the population of allogeneic cells. In some embodiments, the infusion is bolus intravenous infusion. In certain embodiments, the administering comprises administering at least about 1×10⁵ cells of the population of allogeneic cells per kilogram per dose to the human patient. In some embodiments, the administering comprises administering about 1×10⁶ to about 10×10⁶ cells of the population of allogeneic cells per kilogram per dose to the human patient. In some embodiments, the administering comprises administering about 1×10⁶ to about 5×10⁶ cells of the population of allogeneic cells per kilogram per dose to the human patient. In a specific embodiment, the administering comprises administering about 1×10⁶ cells of the population of allogeneic cells per kilogram per dose to the human patient. In another specific embodiment, the administering comprises administering about 3×10⁶ cells of the population of allogeneic cells per kilogram per dose to the human patient. In another specific embodiment, the administering comprises administering about 5×10⁶ cells of the population of allogeneic cells per kilogram per dose to the human patient.

In certain embodiments, the methods of treating WT1-positive multiple myeloma and plasma cell leukemia described herein comprise administering at least 2 doses of the population of allogeneic cells to the human patient. In specific embodiments, the methods of treating WT1-positive multiple myeloma and plasma cell leukemia described herein comprise administering 2, 3, 4, 5, or 6 doses of the population of allogeneic cells to the human patient. In a specific embodiment, the methods of treating WT1-positive multiple myeloma and plasma cell leukemia described herein comprise administering 3 doses of the population of allogeneic cells to the human patient.

In certain embodiments, the methods of treating WT1-positive multiple myeloma and plasma cell leukemia described herein comprise a washout period between two consecutive doses, wherein no dose of the population of allogeneic cells is administered during the washout period. In specific embodiments, the washout period is about 1-8 weeks. In specific embodiments, the washout period is about 1-4 weeks. In specific embodiments, the washout period is about 4-8 weeks. In a specific embodiment, the washout period is about 1 week. In another specific embodiment, the washout period is about 2 weeks. In another specific embodiment, the washout period is about 3 weeks. In another specific embodiment, the washout period is about 4 weeks.

In a specific embodiment, the methods of treating WT1-positive multiple myeloma and plasma cell leukemia described herein comprise administering 3 doses of about 1×10⁶ cells of the population of allogeneic cells per kilogram per dose to the human patient, and a washout period of 4 weeks between two consecutive doses, wherein no dose of the population of allogeneic cells is administered during the washout period. In another specific embodiment, the methods of treating WT1-positive multiple myeloma and plasma cell leukemia described herein comprise administering 3 doses of about 3×10⁶ cells of the population of allogeneic cells per kilogram per dose to the human patient, and a washout period of 4 weeks between two consecutive doses, wherein no dose of the population of allogeneic cells is administered during the washout period. In another specific embodiment, the methods of treating WT1-positive multiple myeloma and plasma cell leukemia described herein comprise administering 3 doses of about 5×10⁶ cells of the population of allogeneic cells per kilogram per dose to the human patient, and a washout period of 4 weeks between two consecutive doses, wherein no dose of the population of allogeneic cells is administered during the washout period.

In a specific embodiment, the administering comprises administering 3 doses to the human patient, each dose being in the range of 1×10⁶ to 5×10⁶ cells of the population of allogeneic cells per kilogram, and wherein the 3 doses are administered about 4 weeks apart from one another. In another specific embodiment, the administering comprises administering 3 doses to the human patient, each dose being in the range of 1×10⁶ to 5×10⁶ cells of the population of allogeneic cells per kilogram, and wherein the 3 doses are administered about 3 weeks apart from one another. In another specific embodiment, the administering comprises administering 3 doses to the human patient, each dose being in the range of 1×10⁶ to 5×10⁶ cells of the population of allogeneic cells per kilogram, and wherein the 2 doses are administered about 3 weeks apart from one another. In another specific embodiment, the administering comprises administering 3 doses to the human patient, each dose being in the range of 1×10⁶ to 5×10⁶ cells of the population of allogeneic cells per kilogram, and wherein the 3 doses are administered about 1 week apart from one another.

In certain embodiments, a first dosage regimen described herein is carried out for a first period of time, followed by a second and different dosage regimen described herein that is carried out for a second period of time, wherein the first period of time and the second period of time are optionally separated by a washout period (for example, about three weeks). Preferably, the second dosage regimen is carried out only when the first dosage regimen has not exhibited toxicity (for example, no grade 3-5 serious adverse events, graded according to NCI CTCAE 4.0).

The term “about” shall be construed so as to allow normal variation.

5.6. Serial Treatment with Different Cell Populations

Also provided herein are methods of treating WT1-positive multiple myeloma or plasma cell leukemia which further comprise, after administering to the human patient the population of allogeneic cells, administering to the human patient a second population of allogeneic cells comprising WT1-specific allogeneic T cells; wherein the second population of allogeneic cells is restricted by a different HLA allele shared with the human patient. In a specific embodiment, the second population of allogeneic cells lacks substantial cytotoxicity in vitro toward antigen presenting cells that are not WT-1 peptide loaded or genetically engineered to (i.e., recombinantly) express one or more WT1 peptides, in the same way as described above for the population of allogeneic cells. In another specific embodiment, the second population of allogeneic cells exhibits substantial cytotoxicity in vitro toward (e.g., exhibits substantial lysis of) antigen presenting cells that are WT-1 peptide loaded, in the same way as described above for the population of allogeneic cells. In another specific embodiment, the second population of allogeneic cells lacks substantial cytotoxicity in vitro toward antigen presenting cells that are not WT-1 peptide loaded or genetically engineered to (i.e., recombinantly) express one or more WT1 peptides, in the same way as described above for the population of allogeneic cells, and exhibits substantial cytotoxicity in vitro toward (e.g., exhibits substantial lysis of) antigen presenting cells that are WT-1 peptide loaded, in the same way as described above for the population of allogeneic cells.

The second population of allogeneic cells can be administered by any route and any dosage/administration regimen as described in Section 5.5.

In certain embodiments, the human patient has no response, an incomplete response, or a suboptimal response (i.e., the human patient may still have a substantial benefit from continuing treatment, but has reduced chances of optimal long-term outcomes) after administering the population of allogeneic cells and prior to administering the second population of allogeneic cells.

In specific embodiments, two populations of allogeneic cells comprising WT1-specific allogeneic T cells that are each restricted by a different HLA allele shared with the human patient are administered serially. In specific embodiments, three populations of allogeneic cells comprising WT1-specific allogeneic T cells that are each restricted by a different HLA allele shared with the human patient are administered serially. In specific embodiments, four populations of allogeneic cells comprising WT1-specific allogeneic T cells that are each restricted by a different HLA allele shared with the human patient are administered serially. In specific embodiments, more than four populations of allogeneic cells comprising WT1-specific allogeneic T cells that are each restricted by a different HLA allele shared with the human patient are administered serially.

### 6. EXAMPLE

Certain embodiments provided herein are illustrated by the following non-limiting examples, which demonstrate that the therapy with a population of allogeneic cells comprising WT1-specific allogeneic T cells according to the invention is effective in treating WT1-positive multiple myeloma and plasma cell leukemia with low or no toxicity.

### 6.1. Example 1. Phase I Clinical Trial to Treat Multiple Myeloma and Plasma Cell Leukemia with WT1-Specific Cytotoxic T Cells

6.1.1. Introduction

We developed a Phase I clinical trial (IRB#12-175) which is designed to treat patients with pPCL, sPCL, and refractory myeloma with allogeneic TCD HSCT (T cell-depleted hematopoietic stem cell transplantation) followed by the administration of donor-derived WT1-specific cytotoxic T cells (WT1 CTLs). The WT1 CTLs can be administered as early as, for example, 6 weeks post TCD HSCT because these T cell lines lack alloreactivity and can therefore be administered much earlier than unmodified donor lymphocytes without inducing GvHD. The early administration of these cells post-allogeneic HSCT in patients with plasma cell leukemia is advantageous as the median progression free and overall survival is as short as 9-12 weeks post-allogeneic HSCT. First results and correlative data of patients treated with this approach are encouraging and the early administration (6-8 weeks post-allogeneic HSCT) of donor-derived WT1-specific T cells in 7 patients treated with these CTLs has shown no side effects including no GvHD up to 7 months post-allogeneic HSCT.

6.1.2. Methods and Materials:

Generation of WT1-specific CTLs

To isolate T cells for sensitization and in vitro expansion, mononuclear cells were initially isolated from heparinized blood or leukapheresed white cell preparations by centrifugation on Ficoll-Hypaque density gradient. After washing, T cells were enriched by initially depleting monocytes by adherence to sterile plastic tissue culture flasks or by the clinical grade CD14 microbeads (Miltenyi) if started from frozen/thawed PBMCs (peripheral blood mononuclear cells). NK cells were also depleted by incubation with clinical grade anti-CD56-microbeads reagent (Miltenyi Biotech). The CD56+ and CD14+ cells were then removed by adherence of the beads in a magnetized sterile column. The T cell enriched cell fractions were then washed and suspended in medium containing 5% prescreened heat-inactivated AB serum in preparation for sensitization.

For sensitization in vitro, autologous cytokine activated monocytes (CAMs) and autologous EBV BLCL prepared as previously described (Doubrovina et al., 2004, Clin Cancer Res 10:7207-7219), were loaded with a pool of 141 overlapping 15-mers spanning the sequence of WT1, each 15-mer being at a concentration of 0.35 μg/ml. The peptides were synthesized by Invitrogen and were certified to be 95% pure and microbiologically sterile. To load the two types of antigen presenting cells (APCs), the pool of nonapeptides, solubilized on DMSO, were added to washed DCs (dendritic cells) or EBV BLCLs (Epstein-Barr Virus-transformed B lymphocyte cell lines) suspended in serum free medium at a concentration of 1×10⁶ cells/ml. These cell mixtures were incubated for 3 hours, then washed with serum free medium and added to the T cells suspended at a concentration of 2×10⁶ T cells/ml in medium containing 5% heat-inactivated human AB serum at an effector T cell to APC ratio of 20:1. Cultures were maintained at 37° C. in an atmosphere of 5% CO₂ in air. At initiation, the cultures were sensitized and resensitized 7 days thereafter with peptide loaded CAMs. Thereafter, peptide loaded EBV BLCLs were used for resensitizations. Resensitizations were performed every week at 4:1 T cell to APC ratio. After 7 days of initial culture, IL2 was added at 3 day intervals to a concentration of 10 IU/ml. IL15 also was added weekly to the CTL culture media at 10 ng/ml.

After 28-35 days of sensitization, if the T cells were cytotoxic and specific they were expanded if required in large scale cultures with IL2 and OKT3 according to a modification of the technique of Dudley and Rosenberg (Dudley and Rosenberg, 2007, Semin Oncol 34:524-531), using irradiated autologous WT1 peptide loaded EBV BLCLs as irradiated feeders.

Quality Assessment of WT1 Peptide Sensitized T Cells Prior to their Release for Use for Adoptive t Cell Therapy

The sensitized T cells were assessed for their specificity and reactivity against WT1 peptides 1) by FACS enumeration of CD3+, CD8+ and CD4+ T cells, and 2) by assessing their cytotoxicity against unmodified and peptide loaded autologous and allogeneic antigen-presenting cells (APC) (such as donor or patient derived PHA stimulated blasts, donor derived dendritic cells and donor derived EBV transformed B cells). T cell mediated cytotoxicity was measured using standard ⁵¹Cr release assays as previously described (Trivedi et al., 2005, Blood 105:2793-2801).

T cell cultures containing the required dose of WT1 peptide sensitized T cells and lacking more than background responses to unloaded donor and recipient cells were considered for cryopreservation and subsequent use for adoptive immunotherapy. They were also tested by standard cultures for microbiological sterility. Mycoplasma tests and endotoxin levels were also obtained.

T cells were considered acceptable for administration if:

1. The viability of the cells is >70%;

2. The identity of the T-cells as derived from the patient's transplant donor is confirmed by HLA typing;

3. The T-cell product is microbiologically sterile, free of mycoplasma and contains <5EU of endotoxin/ml of the T-cell culture at final freeze;

4. The T-cells can specifically lyse >20% WT-1 total peptide pool-loaded autologous donor APC and/or WT-1 total peptide pool-loaded PHA blasts of the patient's genotype;

5. The T-cells lyse <15% unmodified PHA blasts from the T-cell donor (autologous) or the allogenic donor's transplant recipient who is to be treated;

6. The T-cells lyse <15% of HLA mismatched EBVBLCL; and

7. The T cell preparations contain <2% CD19+ B cells.

Quantitation of Functional WT1-Specific T Cells by Intracellular IFN-γ Analyses

Frequencies of WT1-specific T cells were determined at various time points pre and post CTL infusions by quantifying WT1-specific IFN-γ production. The intracellular IFN-γ production assay was performed as previously described (Trivedi et al., 2005, Blood 105:2793-2801; Tyler et al., 2013, Blood 121:308-317). Briefly, peripheral blood mononuclear cells (PBMC; 106) were mixed with either unloaded autologous PBMC or PBMC loaded with pools of overlapping WT1 pentadecapeptides and/or analog peptides at an effector-stimulator cell ratio of 5:1 (Trivedi et al., 2005, Blood 105:2793-2801; Tyler et al., 2013, Blood 121:308-317). Control tubes containing effector cells were incubated separately until the staining procedure. Brefeldin A (Sigma, St Louis, Mo.) was added to nonstimulated and stimulated samples at a concentration of 10 μg/mL. After overnight incubation in a humidified 5% CO₂ incubator at 37° C., staining and analyses were performed as previously described (Trivedi et al., 2005, Blood 105:2793-2801; Tyler et al., 2013, Blood 121:308-317). Cells were stained with an anti-CD3 allophycocyanin (APC)-conjugated antibody, anti-CD8 phycoerythrin (PE)-labeled antibody, anti-CD4 Peridin chlorophyll protein (PerCP)-conjugated antibody, fixed/permeabilized, and then stained with anti-IFN-γ fluorescein isothiocyanate (FITC) (all BD Pharmingen, San Jose, Calif.). Data acquisition was performed with a FACSCalibur flow cytometer with triple lasers for 10-color capability using BD FACSDiva Software (BD Biosciences). Data analyses of T-cell frequencies were performed using FlowJo software (Tree Star Inc, Ashland, Oreg.).

To determine the WT1-derived epitope, we assessed the capacity of T-cells to produce intracellular IFN-γ in response to PBMCs pulsed with one of each of the 22 pentadecapeptide pools. Thereafter, single pentadecapeptides of positive pools were tested to induce intracellular IFN-γ. HLA-restriction was then analyzed by T-cell cytotoxicity for their capacity to lyse peptide-pulsed or control target-cells using a standard ⁵¹Cr cytotoxicity assay as previously described (Trivedi et al., 2005, Blood 105:2793-2801). Target cells included patient-derived plasma cell-containing specimen (peripheral blood or bone marrow), patient PHA blasts and EBV-BLCLs of known HLA-type which were either pulsed with relevant or irrelevant peptides as previously described (Trivedi et al., 2005, Blood 105:2793-2801; Dudley and Rosenberg, 2007, Semin Oncol 34:524-531).

Determination of WT1 Peptide-Specific Frequencies by MHC-Tetramer Analyses

WT1-specific T-cell frequencies were also quantified at the same time points in patients expressing the HLA alleles A*0201 and A*0301 by staining with the appropriate A*0201/RMF and A*0301/RMF major histocompatibility complex (MHC)-tetramers as previously described. In brief, PBMCs were stained with 25 μg/mL PE-labeled tetrameric complex, 3 μL of monoclonal anti-CD3 phycoerythrincyanin-7 (PE-Cy7), 5 μL of anti-CD8 PerCP, 5 μL of anti-CD45RA APC, and 5 μL of anti-CD62L FITC (all BD Bioscience) for 20 minutes at 4° C. Appropriate control stains with HLA-mismatched tetramers were also performed. The stained cells were subsequently washed, resuspended in fluorescence-activated cell sorting (FACS) buffer (PBS++ with 1% BSA and 0.1% sodium azide). Data acquisition was performed with a FACSCalibur flow cytometer with triple lasers for 10-color capability using BD FACSDiva Software (BD Biosciences). Data analyses of T-cell frequencies were performed using FlowJo software (Tree Star Inc, Ashland, Oreg.).

Analysis of In Vitro Cytotoxicity

A standard 4 hour ⁵¹Cr labeled cytotoxicity assay was utilized to assess in vitro efficacy. Target cells for lysis included HLA-A*02 positive human myeloma cell lines (previously identified via flow cytometry) and autologous and HLA-matched host (for donor derived T cells) CD138 myeloma cells, positively selected via magnetic beads. HLA-A*02 negative human myeloma cell lines and autologous (or matched host in the case of donor derived T cells) peripheral blood mononuclear cells were used as negative controls.

T Cell-Depleted Hematopoietic Stem Cell Transplantation

All patients were conditioned for allogeneic T cell-depleted hematopoietic stem cell transplantation (TCD HSCT) with busulfan (Busulfex®) (0.8 mg/Kg/dose Q6H×10 doses), melphalan (70 mg/m²/day×2 doses) and fludarabine (25 mg/m²/day×5 doses). Doses of busulfan and melphalan were adjusted according to ideal body weight, busulfan was adjusted according to first dose pharmacokinetic studies and doses of fludarabine was adjusted according to measured creatinine clearance. Patients also received ATG (Thymoglobulin®) prior to transplant to promote engraftment and to prevent graft-versus host disease post transplantation.

The preferred source of stem cells were peripheral blood stem cells (PBSCs) mobilized by treatment of the donor with G-CSF for 5-6 days. PBSCs were isolated, and T cells depleted by positive selection of CD34+ progenitor cells, using the CliniMACS Cell Selection System. The CD34+ T cell-depleted peripheral blood progenitors were then administered to the patients after they completed cytoreduction. No drug prophylaxis against GvHD was administered post transplant. All patients also received G-CSF post-transplant to foster engraftment. The patients also had a hematopoietic stem cell transplant donor who consented to donate additional blood to generate the WT1-specific cytotoxic T cells.

6.1.3. Results:

The trial enrolled patients with primary plasma cell leukemia (pPCL) or secondary plasma cell leukemia (sPCL), and relapsed/refractory multiple myeloma. On protocol, patients underwent allogeneic T-cell depleted hematopoietic stem cell transplantation (TCD HSCT) followed by the intravenous administration of donor-derived WT1-specific cytotoxic T cells (WT1 CTLs). The WT1 CTLs were administered as early as 6 weeks post allogeneic TCD HSCT because these T cell lines lost alloreactivity though the sensitization during culture and we hypothesized that these cells could therefore be administered much earlier than unmodified donor lymphocytes without inducing GvHD. The early administration of these cells in patients with PCL or relapsed/refractory MM was carried out, since the median progression free and overall survival is short.

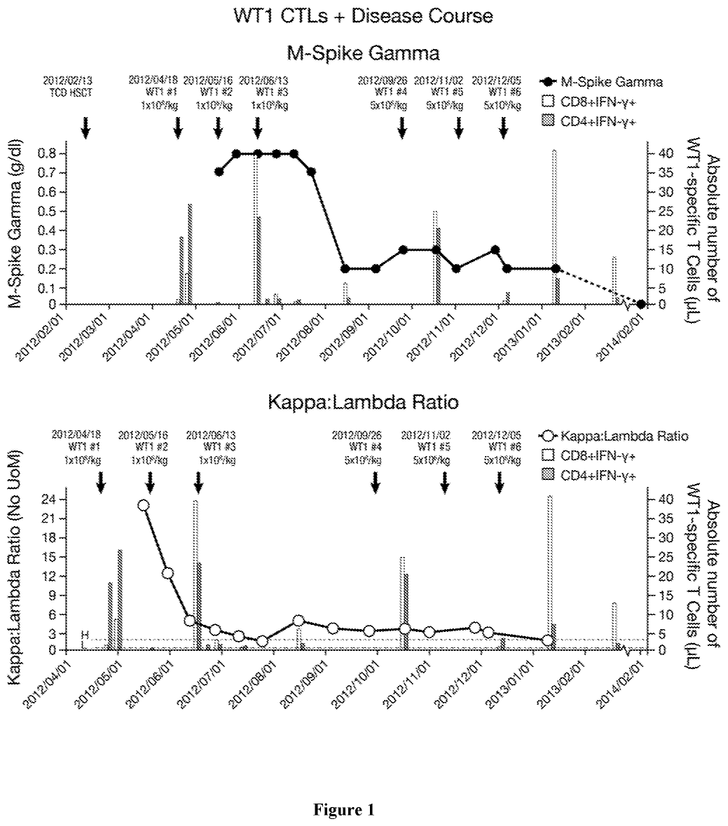

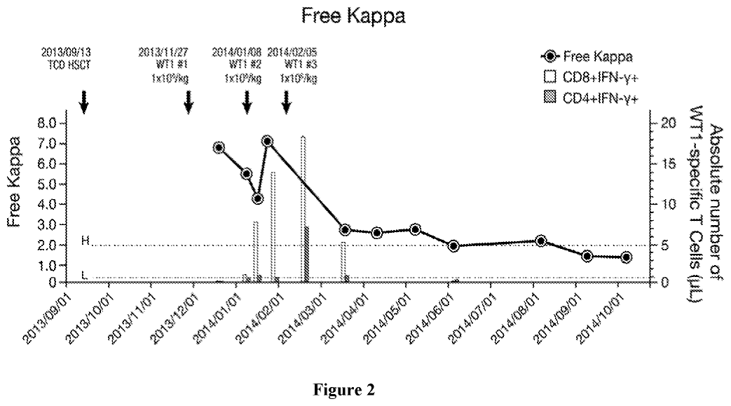

We have registered 11 patients onto our protocol and 7 patients have been treated with donor-derived WT1-specific CTLs post allogeneic TCD HSCT. Based on the aggressive biology of PCL, 4 patients progressed and expired prior to the administration of the WT1-specific CTLs and were taken off the study. For this trial, the WT1-specific T cells were generated in our GMP facility by sensitizing donor lymphocytes with antigen-presenting cells that were pulsed with a peptide pool of overlapping pentadecapeptides over spanning the WT1 protein. WT1 CTLs were given at 1×10⁶/kg/week, 3×10⁶/kg/week or 5×10⁶/kg/week×3 doses at each dose level and administered at 4 weekly intervals starting at 6-8 weeks post transplantation. No side effects including GvHD were observed in these patients. We have observed impressive clinical responses in these patients, and have analyzed WT1-specific T-cell responses associated with increments of both CD8+ and CD4+ WT1-specific T cell in blood and bone marrow of these patients. Two examples are demonstrated in FIGS. 1 and 2.

The patient treated in FIG. 1 underwent allogeneic TCD HSCT for sPCL refractory to salvage chemotherapy with VDT-PACE (a combination chemotherapy regimen with bortezomib, dexamethasone, thalidomide, cisplatin, doxorubicin, cyclophosphamide, and etoposide). As demonstrated, the patient still had significant disease following TCD HSCT with an M-spike of 0.8 g/dl and a kappa: lambda ratio of 24. We analyzed WT1-specific T-cell frequencies by intracellular IFN-γ analyses, as described above, and plotted the absolute numbers of CD8+ and CD4+ WT1-specific T cells following WT1-specific T-cell infusions. As shown in FIG. 1, the disease markers decreased while the absolute numbers of CD8+ and CD4+ cells WT1-specific CTLs significantly increased. This patient developed a complete remission that lasted greater than 2 years.

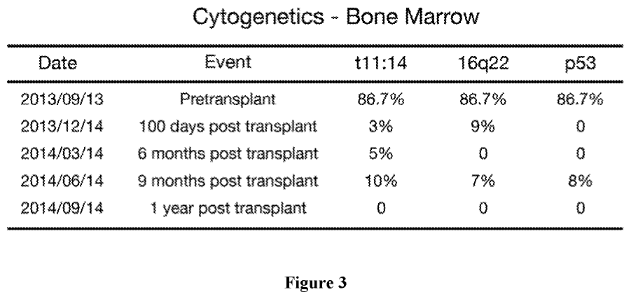

FIG. 2 shows the results obtained following allogeneic TCD HSCT and subsequent infusion of donor-derived WT1-specific CTLs in a patient with pPCL refractory to previous treatments, including 5 cycles of RVD (a combination chemotherapy regimen with lenalidomide, bortezomib, and dexamethasone), 2 cycles of VDT-PACE, and autologous hematopoietic stem cell transplantation with melphalan 200 mg/m² conditioning regimen. This patient still had residual disease as measured by free kappa light chains following autologous stem cell transplant and as demonstrated, his specific disease marker was still at elevated levels post allogeneic TCD HSCT but declined to normal level following the administration of 3 doses of WT1-specific CTLs while he developed CD8+ and CD4+ WT1-specific T cell frequencies, as measured by intracellular IFN-γ analyses, following the CTL infusions. This patient has been in CR (complete response) for >1½ years. Interestingly, as shown in FIG. 3, his high risk cytogenetics, measured in the enriched plasma cell population from his bone marrow, also cleared following the WT1-specific CTL infusions.

Another patient with sPCL was treated and achieved a complete remission following an induction chemotherapy followed by an autologous hematopoietic stem cell transplantation. This patient underwent an allogeneic TCD HSCT from an unrelated donor 3 months later and received subsequently 3 doses of donor-derived WT1 CTLs. This patient with sPCL has been in complete remission for 2 years.

In addition, 4 patients with relapsed/refractory multiple myeloma were treated with allogeneic TCD HSCT followed by the administration of donor-derived WT1 CTLs. All of these patients had failed to respond to multiple lines of treatment including combination therapy with lenalidomide and bortezomib and autologous hematopoietic stem cell transplantation. One of these patients has developed a partial response and has continued to have a partial response at 18 months post-allogeneic HSCT. Two of these patients have developed stable disease, both for 19 months post-allogeneic HSCT. Only one of these patients developed aggressive progression of disease with development of sPCL 7 months post-allogeneic HSCT and 5 months post-administration of WT1 CTLs and subsequently succumbed of sPCL refractory to further chemotherapeutic combinations.

### 6.2. Example 2. Assessment of the Efficacy of Third-Party WT1-Specific Cytotoxic T Cells Using 11929 and L363 Models of Multiple Myeloma/Plasma Cell Leukemia

6.2.1. Synopsis

6.2.1.1. Studied Period

For greater than 3 months.

6.2.1.2. Purpose

To assay the anti-multiple myeloma (MM)/plasma cell leukemia (PCL) efficacy of ATA 520 in mouse models of disseminated disease when deployed in a third-party setting schema.

6.2.1.3. Animals

NOD/Shi-scid/IL-2Rγnull (NOG) female mice age 5-6 weeks.

6.2.1.4. Test Articles

T cell line library: ATA 520. T cell lines from ATA 520 selected by being restricted to HLA allele shared with MM target cell line.

H929 MM target cell line matched on HLA A03:01 with T cell line designated Lot #3 from ATA 520.

L363 MM target cell line matched on HLA C07:01 with T cell line designated Lot #4 from ATA 520.

6.2.1.5. Methods

MM cell lines were HLA typed and matched to appropriately restricted T cell lines of ATA 520 as indicated in Test article information. Two 3-arm in vivo efficacy studies with selected Multiple Myeloma models (cell line-derived xenografts, “CDX”) were conducted with L363 and H929 cell lines. Assessment of the anti-tumor activity of intravenously injected T cells at two different weekly doses (2×10⁶ cells per mouse and 10×10⁶ cells per mouse, respectively) in monotherapy was performed using in life imaging with a fluorescently labeled anti-CD138 antibody.

Experiments comprised 8 animals/group receiving tumor implants intravenously (injection of 5×10⁶ cells per animal). Minimum group size at randomization was 7 animals/group. The scheduled treatment period was 5 weeks. As a reference, a vehicle control group was included (vehicle: phosphate-buffered saline).

Body weight determination (twice weekly) and in vivo disease imaging (“IVI”, once weekly, using anti-CD138 antibody) were performed.

Sternum, hind-legs, liver, and spleen samples were taken as available for later analyses.

6.2.1.6. Results and Conclusions

Following 5 dose cycles of the ATA 520 T cell lines in MM/PCL diseased mice, treatment resulted in maximal disease growth inhibition of 51.9% in H929 models and 18.2% in L363 models compared to the vehicle control over the treatment period (p<0.002 and p<0.01, respectively, by one-way ANOVA). The level of disease control between low and high dose groups was not significantly different across these two studies.

Using these two models as a preclinical proxy for deployment of ATA 520 in a third-party fashion (ATA 520 is partially matched to non-related target cells by HLA), this study establishes the capacity for ATA 520 to significantly inhibit tumor growth of disseminated multiple myeloma and plasma cell leukemia.

6.2.2. List of Selected Abbreviations and Definitions

| Abbreviation | Expanded term |
|---|---|
| NOG | NOD/Shi-scid/IL-2Rγnull |
| TGI | Tumor growth inhibition |
| ANOVA | Analysis of variance |
| IACUC | Institutional Animal Care and Use Committee |
| IV | intravenous |
| MM | multiple myeloma |
| QD | Quaque die, once daily |
| SEM | Standard error of the mean |
| MM/PCL | Multiple Myeloma/Plasma Cell Leukemia |
| HLA | Human Leukocyte Antigen |

6.2.3. Introduction

ATA 520 is a library of different T cell lines specific to WT-1 epitopes presented by context-specific HLAs. When a T cell line of ATA 520 is used, having a restriction matched to a WT-1 epitope presented on an HLA allele found on allogeneic target cells, the T cell line facilitates degranulation and T cell induced elimination of the target cell. WT1 is a transcription factor commonly found in nuclear regions of cells, when expressed. Expression of WT1 is common in many solid and hematopoietic malignancies. Clinical data has been presented for use of T cell lines of ATA 520 in an allo-setting post transplant in MM and PCL populations.

To model the use of ATA 520 cell lines in a third-party setting in a similar treatment population, this study uses NOD/Shi-scid/IL-2Rγnull (NOG) mice harboring human cell xenografts of MM/PCL as a proxy for a patient with MM/PCL. The diseased cells in this proxy are subjected to comprehensive HLA typing and compared to ATA 520 T cell lines with restrictions annotated to one HLA. ATA 520 T cell lines were selected based on matching to one HLA allele found on the target cells, constituting a third-party model for treatment selection.

Therefore, this study was conducted to assay the antitumor efficacy of ATA 520 when deployed in a third-party setting in in vivo models of MM/PCL.

6.2.4. Objectives

This study was conducted to assay the antitumor efficacy of ATA 520 when deployed in a third-party setting in in vivo models of MM/PCL.

6.2.5. Test Animals

H929 Models

24 female NOG mice

Source: Taconic

Age range at start of the study: 5-6 weeks

L363 Models

24 female NOG mice

Source: Taconic

Age range at start of the study: 5-6 weeks

6.2.6. Test Animal Housing and Care

Female NOG mice 5-6 weeks of age were housed at the Oncotest/CRL Vivarium. The mice were kept in a barrier system with controlled temperature (70°±10° F.), humidity (50%±20%) and a lighting cycle of 12 hr light/12 hr dark. Mice were housed in isolator cages (5 mice per cage) and had free access to standard pellet food and water during the experimental period. All mice were treated in accordance with guidelines outlined by the Oncotest/CRL Institutional Animal Care and Use Committee (IACUC).

6.2.7. Study Materials

ATA 520 T cell lines (including Lot #3 and Lot #4) were synthesized at Memorial Sloan Kettering Cancer Center (MSKCC), and maintained as concentrated solutions and stored in liquid nitrogen until use. ATA 520 was generated using the methods described in Section 6.1.2.

6.2.8. Study Design

The study protocol is summarized in Table 1.

Female NOG mice 5-6 weeks of age were intravenously (IV) implanted with 5×10⁶ H929 or L363 cells. Weekly imaging was conducted with IV administration of hCD138Ab-Alexa750 to track engraftment status using an IVIS® imaging system. When mean whole body measurements were evident (˜14-17 days after inoculation), mice were distributed into three groups so as to normalize resultant mean signal per group. Minimum group size at randomization was 7 animals/group. Mice then received either 10 ml/kg vehicle (i.e., phosphate-buffered saline), or a ATA 520 T cell line at 2×10⁶ or 10×10⁶ cells/mouse (i.e., 5×10⁶ cells/ml or 25×10⁶ cells/ml, with a volume of 0.4 ml per mouse) on a Q7D (i.e., once every 7 days)×5 schedule. Mice were imaged every 7 days during the dosing scheme to evaluate disease burden. Body weight was determined twice weekly. Sternum, hind-legs, liver, and spleen samples were taken as available for later analyses.

TABLE 1
Summary of Study Design

| Group | | ATA 520 | Dose | Dosing | | | | |
|---|---|---|---|---|---|---|---|---|
| # | Tx | Dose | Volume^{b} | Days | Route | Vehicle | n | Monitoring |
| 1 | Vehicle | — | 20 ml/kg | Q7Dx5 | IV | PBS | 8 | IVI |
| | | | | | | | | Q7Dx6^{a} |
| 2 | ATA 520 | 2 × 10⁶ | 20 ml/kg | Q7Dx5 | IV | PBS | 8 | IVI |
| | Low Dose | cells/mouse | | | | | | Q7Dx6 |
| 3 | ATA 520 | 10 × 10⁶ | 20 ml/kg | Q7Dx5 | IV | PBS | 8 | IVI |
| | High Dose | cells/mouse | | | | | | Q7Dx6 |

^{a}Q7Dx6 means once every 7 days for 6 times.
^{b}Mice were assumed to be 20 gram for dose calculation purposes.

6.2.9. Experimental Procedures

6.2.9.1. HLA Testing and ATA 520 Cell Line Selection

Frozen cell pellets of H929 and L363 target cell lines were HLA characterized using Tier 1 resolution sequencing (Table 2). Generally, gDNA preparations were made from cell pellets using Qiagen kits. Typing was then conducted by PCR-sequence specific oligonucleotides (PCR-SSOP) to resolve major allele groups to 4 digits, with some degeneracy (e.g., HLA-A*23:01/03/05/06).

The genomic DNA was amplified using PCR, then incubated with a panel of different oligonucleotide probes using Luminex xMAP® technology; each oligonucleotide has distinctive reactivity with different HLA-types.

Resultant HLA characteristics for each of the two target cell lines were then compared to restriction characteristics within the library of AT-520 to identify matching T cell lines for each target cell line (Table 3). One matching T cell line was then used in the treatment schema for mice harboring target-specific MM/PCL disease for each of the two target cell lines.

6.2.9.2. Dose Formulation and Administration

Frozen vials of concentrated selected T cell lines of ATA 520 were gently thawed in a 37° C. water bath. The concentrated solution was gently agitated and made homogeneous by pipetting repeatedly using a 1 ml pipet. The ATA 520 T cell lines were then diluted in PBS +10% human albumin into a dosing stock with a concentration of 25×10⁶ cells/ml for the high dose group, or 5×10⁶ cells/ml for the low dose group. Dosing stocks were prepared fresh each dose day.

Animals were dosed once weekly for 5 weeks (Q7D×5) by intravenous injection.

6.2.9.3. In Vivo Antitumor Efficacy

5×10⁶ MM/PCL cells were implanted into NOG mice 12-17 days prior to initiation of treatment. On Day 0 of dosing, female NOG mice were administered either vehicle or ATA 520 T cell line at two distinct doses (as stipulated in Table 1) for 5 weekly cycles. Disease burden was monitored by administering hCD138-Alexa750 IV and measuring whole body fluorescence as a proxy of tumor burden during the treatment period. Images were analyzed and a sum of dorsal and ventral signals was quantified and recorded. Mean and standard error for whole body signals were calculated for each treatment group for each imaging session. Mean whole body signals ±standard error of the mean (SEM) were plotted against treatment day to represent tumor growth kinetics associated with each group over the duration of the study. To calculate tumor growth inhibition (TGI) at the end of study, percent inhibition of whole body signal was calculated for each mouse compared to a vehicle control group. Mean percent inhibition ±SEM for each group was generated. The above calculations were conducted, with standard errors tracked, using GraphPad Prism v.6.0c. Resultant group TGI values were analyzed by one-way analysis of variance (ANOVA) and Tukey's multiple comparisons test.

6.2.9.4. Statistical Methods

All comparative intensity and TGI calculations were performed using GraphPad Prism v6.0c. Group TGI values were analyzed by one-way ANOVA and Tukey's multiple comparisons test.

6.2.10. Data and Results

6.2.10.1. HLA Typing and ATA 520 Restriction Matching

Results from Tier 1 level HLA typing of MM/PCL target cells by PCR is shown in Table 2.

TABLE 2
HLA Typing of L363 and H929 Target Cells*

| Allele | | |
|---|---|---|
| Family | L363 | H929 |
| HLA-A | *02:01/01Q/01L/09/43N/66/75/83N/89/97/132/134/ | *03:01/01N/20/21N/26/37/45/78/112/118/ |
| | 140/241/252/256/266/291/294/305N/327/329/356N/ | 129N/132/134/162N/182/184/188/195/197N/ |
| | 357/397/411/446/455/468N/469/48- | 209 |
| | 483/491/501N/502/525N/530/533/538/539/542/552 | |
| HLA-A1 | *31:01/14N/23/46/48/55/56/59/71/72/81/83/6 | *24:02/02Q/09N/11N/40N/76/79/83N/144/ |
| | | 150/153/154/155N/163N/183N/231/249- |
| | | 251/263- |
| | | 272/275/276/279/280/282/283/287/292 |
| HLA-B | *07:02/44/49N/58/59/61/104/120/128- | *07:02/07/44/49N/58/59/61/104/120/128- |
| | 130/156/161N/169/212/217/221/224/230/231N/233/ | 130/156/161N/169/212/217/221/224/230/ |
| | 240/241 | 231N/233/234/240/241 |
| HLA-B1 | *40:01/55/141/150/151/179/221/236/241/247/264/ | *18:01/14/53/73/81/89/99/100/103 |
| | 272/273/277/278/286N | |
| HLA-C | *03:04/100/101/105/106/186/193/208N/211/212- | *07:01/06/18/19/52/103/131/153/166/301/ |
| | 213/218/219/235/236/239/252/256/257/259 | 303/310/311/313/331/337/343/362/366/369/ |
| | | 371/377/385/386/407/408 |
| HLA-C1 | *07:02/50/66/74/159/160/167/195/245/305/306/308/ | *07:02/27/50/66/74/159/160/167/195/245/ |
| | 319-321/327/334/339- | 305/306/308/312/316/319- |
| | 341/344/345/348/349/350N/359/363/368/374/381- | 321/325/327/333/334/339- |
| | 384/389/391/392/393N/398/400/401/405 | 341/344/345/347N/348/349/350N/359/363/ |
| | | 368/372/374/376/381- |
| | | 384/388/389/392/393N/396- |
| | | 398/400/401/405/409 |

The HLA typing data in Table 2 was cross referenced to the HLA restrictions of WT-1 specific CTLs in the ATA 520 library to identify T cell lines of ATA 520 compatible with the HLA allele profiles of the target cells by matching restriction to one allele found on the target cells. T cell lines of the ATA 520 library with a restriction matching one allele on at least one of the target cells are shown in Table 3.

TABLE 3
T Cell Lines of ATA 520 Compatible
with Target Cell HLA Profiles

| | | Matching | |
|---|---|---|---|
| | | Allele | |
| Unique | | Family | |
| Lot ID | WT-1 HLA Restriction (CTLs only) | H929 | L363 |
| Lot # 1 | A 0201 | | A |
| Lot # 2 | A 0201 | | A |
| Lot # 3 | A 0301/01N | A | |
| Lot # 4 | C 0701/06/18 | C | C |
| Lot # 5 | A 2402 B 3503 | A | |
| Lot # 6 | C 0701/06/18 | C | C |
| Lot # 7 | A 0201 C 0401 | | A |
| Lot # 8 | A 0201 C 1203 DRB1 1302 DRB1 1104 | | A |

Table 3 depicts a number of ATA 520 T cell lines (cell line identifiers indicated in the first column) whose restriction matches at least one HLA allele expressed on H929 or L363 target cells. ATA 520 cell line restrictions are listed in the 4th column, and the right two columns indicate which allele family found in the target cells match the indicated restriction for each ATA 520 T cell line. The two ATA 520 T cell lines selected for treatment of mice in this study were Lot #3 and Lot #4. T cell line W01-D1-136-10 was selected to treat H929 diseased mice based on the matched restriction to HLA A03:01 allele found in H929. T cell line W01-D1-088-10 was selected to treat L363 diseased mice based on the matched restriction to HLA C07:01 allele found in L363.

6.2.10.2. Clinical Observations

Throughout the dosing period, the animals were observed for any clinically relevant abnormalities and unusual behaviors and reactions. There were no adverse clinical observations noted during the in-life portion of this study.

6.2.10.3. In Vivo Efficacy

Group MM burden for H929-bearing mice are presented in

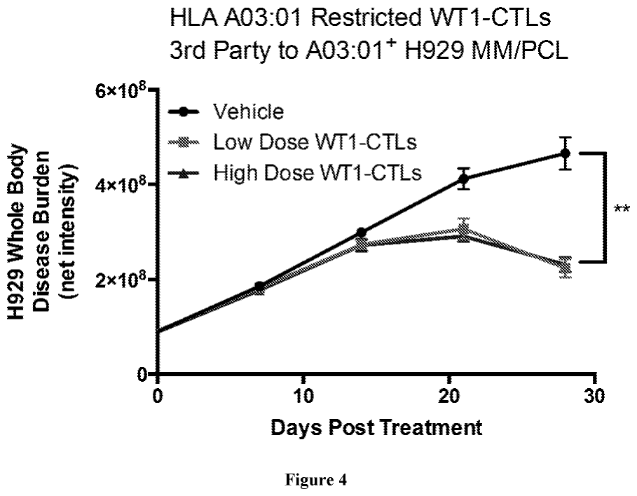

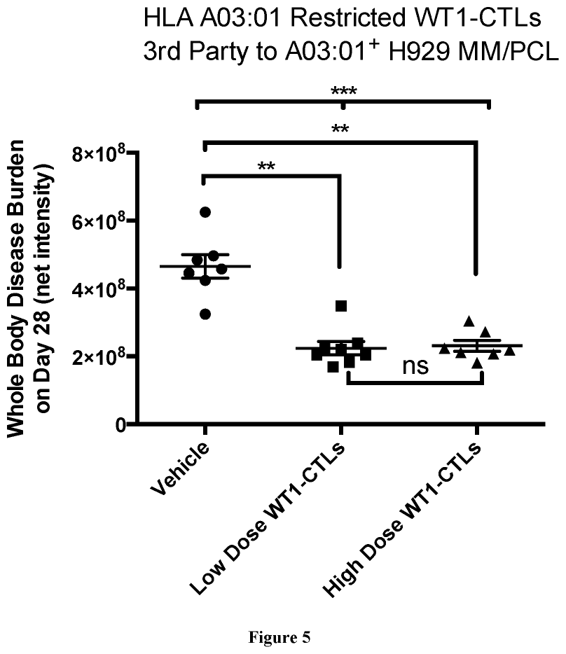

Table 4, and are also presented graphically as a plot and with raw radiance values for each group tracked in FIG. 4. Grouped analysis is also shown for Day 28 in FIG. 5.

TABLE 4
Whole-body MM Burden Fold Change and SEM for H929

| | Vehicle | | Low Dose | | High Dose | |
|---|---|---|---|---|---|---|
| Days Post Treatment | Mean | SEM | Mean | SEM | Mean | SEM |
| 0 | 9.066E+07 | 5.597E+06 | 9.176E+07 | 2.512E+06 | 8.893E+07 | 1.983E+06 |
| 7 | 1.859E+08 | 9.940E+06 | 1.818E+08 | 3.318E+06 | 1.770E+08 | 5.189E+06 |
| 14 | 2.992E+08 | 1.069E+07 | 2.736E+08 | 8.160E+06 | 2.721E+08 | 1.237E+07 |
| 21 | 4.118E+08 | 2.195E+07 | 3.065E+08 | 2.181E+07 | 2.908E+08 | 1.157E+07 |
| 28 | 4.653E+08 | 3.413E+07 | 2.238E+08 | 1.957E+07 | 2.310E+08 | 1.599E+07 |

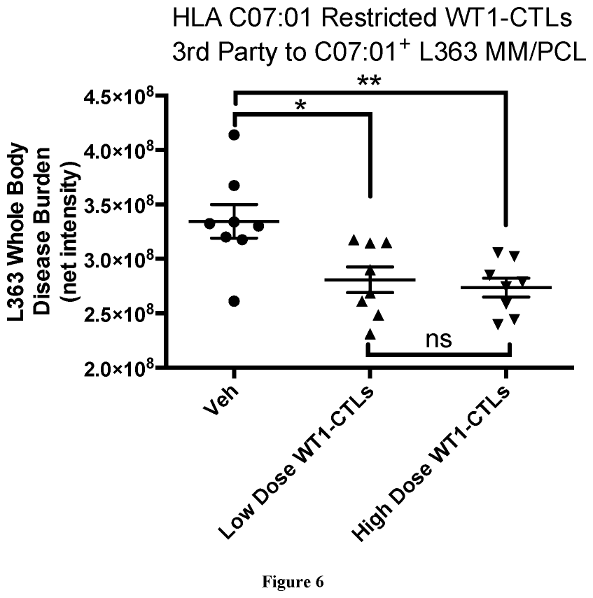

Group MM burden for L363-bearing mice on Day 21, as both mean and individual values, are presented in FIG. 6.

Following 5 dose cycles of the selected ATA 520 T cell line in MM/PCL diseased mice, treatment resulted in maximal disease growth inhibition of 51.9% in H929 models and 18.2% in L363 models compared to the vehicle control over the treatment period (p<0.002 and p<0.01, respectively, by one-way ANOVA). The level of disease control between low and high dose groups was not significantly different across these two studies.

6.2.11. Conclusions

The antitumor efficacy of a library of T cell lines, designated ATA 520, was examined in two orthometastatic xenograft models of multiple myeloma/plasma cell leukemia treated in a third-party setting. Target cells were HLA typed and matched independently to two distinct ATA 520 T cell lines based on restriction of the T cell line to an HLA allele expressed on target cells. In the two models of third-party ATA 520 treatment of MM/PCL, single-agent ATA 520 demonstrated significant tumor growth inhibition under both high and low dose regimens. There was no significant difference in the efficacy observed between high and low dose regimens in both studies. Two independent ATA 520 T cell lines, each restricted by a different HLA allele, significantly inhibited the growth of their respectively matched disease target cells in the two models of third-party treatment.

These results demonstrate potent antitumor activity of ATA 520 T cell lines in advanced MM/PCL models, and demonstrate the feasibility for using a similar treatment approach with third-party derived ATA 520 T cell lines matched to patients by a restricting allele of the ATA 520 T cell line (associated with its activity).

### 7. INCORPORATION BY REFERENCE

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A method of treating WT1 (Wilms Tumor 1)-positive multiple myeloma in a human patient in need thereof, comprising administering to the human patient a population of allogeneic cells comprising WT1-specific allogeneic T cells, wherein the population of allogeneic cells lacks substantial cytotoxicity in vitro toward antigen presenting cells that are not WT1 peptide-loaded or genetically engineered to express one or more WT1 peptides.

2-10. (canceled)

11. The method of claim 1, wherein prior to the administering of the population of allogeneic cells, the human patient has been administered a therapy for multiple myeloma that is different from said population of allogeneic cells, and wherein the multiple myeloma is refractory to the therapy or relapses after the therapy.

12-13. (canceled)

14. The method of claim 11, wherein the therapy is an autologous hematopoietic stem cell transplantation (HSCT).

15. The method of claim 14, wherein the first dose of the population of allogeneic cells is administered on the day of, or up to 12 weeks after, the autologous HSCT.

16-17. (canceled)

18. The method of claim 11, wherein the therapy is an allogeneic HSCT.

19. (canceled)

20. The method of claim 18, wherein the population of allogeneic cells is derived from a third-party donor that is different from the donor of the allogeneic HSCT.

21. The method of claim 18, wherein the first dose of the population of allogeneic cells is administered on the day of, or up to 12 weeks after, the allogeneic HSCT.

22-30. (canceled)

31. The method of claim 1, wherein the first dose of the population of allogeneic cells is administered within 12 weeks after the diagnosis of the multiple myeloma.

32. (canceled)

33. The method of claim 1, wherein said administering of the population of allogeneic cells does not result in any graft-versus-host disease (GvHD) in the human patient.

34. A method of treating WT1-positive plasma cell leukemia in a human patient in need thereof, comprising administering to the human patient a population of allogeneic cells comprising WT1-specific allogeneic T cells, wherein the population of allogeneic cells lacks substantial cytotoxicity in vitro toward antigen presenting cells that are not WT1 peptide-loaded or genetically engineered to express one or more WT1 peptides.

35-45. (canceled)

46. The method of claim 34, wherein prior to the administering of the population of allogeneic cells, the human patient has been administered a therapy for plasma cell leukemia that is different from said population of allogeneic cells, and wherein the plasma cell leukemia is refractory to the therapy or relapses after the therapy.

47-48. (canceled)

49. The method of claim 46, wherein the therapy is an autologous HSCT.

50. The method of claim 49, wherein the first dose of the population of allogeneic cells is administered on the day of, or up to 12 weeks after, the autologous HSCT.

51-52. (canceled)

53. The method of claim 46, wherein the therapy is an allogeneic HSCT.

54. (canceled)

55. The method of claim 53, wherein the population of allogeneic cells is derived from a third-party donor that is different from the donor of the allogeneic HSCT.

56. The method of claim 53, wherein the first dose of the population of allogeneic cells is administered on the day of, or up to 12 weeks after, the allogeneic HSCT.

57-62. (canceled)

63. The method of claim 34, wherein the first dose of the population of allogeneic cells is administered within 12 weeks after the diagnosis of the plasma cell leukemia.

64. (canceled)

65. The method of claim 34, wherein said administering of the population of allogeneic cells does not result in any GvHD in the human patient.

66. The method of claim 1, wherein the population of allogeneic cells is restricted by an HLA allele shared with the human patient.

67-88. (canceled)

89. The method of claim 1, wherein the administering is by infusion of the population of allogeneic cells.

90-91. (canceled)

92. The method of claim 1, wherein the administering comprises administering about 1×10⁶ to about 5×10⁶ cells of the population of allogeneic cells per kilogram per dose to the human patient.

93-105. (canceled)

106. The method of claim 1, further comprising, after administering to the human patient the population of allogeneic cells, administering to the human patient a second population of allogeneic cells comprising WT1-specific allogeneic T cells, wherein the second population of allogeneic cells is restricted by a different HLA allele shared with the human patient.

107. The method of claim 34, wherein the population of allogeneic cells is restricted by an HLA allele shared with the human patient.

108. The method of claim 34, wherein the administering is by infusion of the population of allogeneic cells.

109. The method of claim 34, wherein the administering comprises administering about 1×10⁶ to about 5×10⁶ cells of the population of allogeneic cells per kilogram per dose to the human patient.

110. The method of claim 34, further comprising, after administering to the human patient the population of allogeneic cells, administering to the human patient a second population of allogeneic cells comprising WT1-specific allogeneic T cells, wherein the second population of allogeneic cells is restricted by a different HLA allele shared with the human patient.
